(19) United States
(12) Patent Application Publication
(10) Pub. No.: US 2008/0213125 A1
(43) Pub. Date: Sep. 4, 2008

(54) Apparatus and Method for Using Ozone as a Disinfectant

(75) Inventors: Nigel Boast, Kelowna (CA); Doug Heselton, Surrey (CA); Jim Hudson, Delta (CA); Sharma Manju, Vancouver (CA)

Correspondence Address: Robert E Krebs;Thelen Reid & Priest LLP, P O Box 640640, San Jose, CA, 95164-0640, US

(73) Assignee: Huawei Technologies Co. LTD., Shenzhen, Guangdong Province (CN)

(21) Appl. No.: 10/593,377

(22) PCT Filed: Mar. 18, 2005
(86) PCT No.: PCT/CA05/00412

§ 371 Date: Feb. 4, 2008

Related U.S. Application Data
(60) Provisional application No. 60/553,937, filed on Mar. 18, 2004.
(60) Provisional application No. 60/625,101, filed on Nov. 5, 2004.
(60) Provisional application No. 60/656,888, filed on Mar. 1, 2005.

(51) Int. Cl. A61L 9/015; A61L 2/20; A61L 2/28

(52) U.S. Cl. 422/2; 422/83; 422/28

(57) ABSTRACT

A method of sterilizing a closed environment is provided in which an ozone generator is placed into the closed environment; it then generates ozone to a predetermined ozone concentration and increases the humidity of the closed environment. The ozone concentration is maintained at the predetermined ozone concentration for a predetermined period of time, and after the period of time has expired, the ozone is depleted. When the ozone concentration is reduced to a predetermined safe level, the ozone generator signals.

This application claims the benefit of U.S. Provisional Patent Application Nos. 60/553,937 filed Mar. 18, 2004; unassigned, filed Nov. 8, 2004; and unassigned filed Mar. 1, 2005, which are hereby incorporated by reference.

### FIELD OF THE INVENTION

This invention relates to tools and methods for sterilizing closed environments, and more particularly to the use of ozone to sterilize a room.

### BACKGROUND OF THE INVENTION

People traveling around the world have resulted in the rapid spread of emerging viruses and other diseases. If a disease becomes prevalent in a particular city, it can quickly spread internationally due to travel of the originating city's inhabitants. Once the disease is identified and infected individuals isolated, the disease has often already spread to high-density municipal areas, potentially in other countries.

An example of such a disease is found in the rapid spread of Severe Acute Respiratory Syndrome (SARS) which has a high morbidity and mortality rate and can be difficult to treat. It is very difficult to screen infected people and prevent them from spreading the disease. In particular, the spread of such diseases poses a high risk to the hospitality industry, and can lead to reduced earnings and share prices of public companies in the hospitality sector. The aggressive spread of SARS from Asia to other countries including the United States and Canada has challenged the airline, hospitality and tourism industries as well as hospitals. The spread of SARS also had a negative impact on affected countries' economies, including that of major cities such as Toronto.

SARS is not the only virus of concern. A variety of airborne, gastro enteric and enteric viruses, including varicella zoster (chicken pox), measles virus, rhinovirus (cold), influenza virus (flu), poliovirus, rotavirus, hepatitis A, Norwalk virus, adenovirus, and emerging viruses all represent risks of contagion and infection. The spread of bacterial infections and fungus can also be of significant concern, particularly when drug-resistant varieties occur.

Such diseases are also of concern in the health care sector. For example, clostridium difficile (a human pathogenic bacterium of the gut) is very difficult to remove when infected individuals are kept at a hospital. Health care workers and future patients may be put at risk in such situations.

Ozone has long been recognized as an effective biocide (a biochemical disinfectant), and is also a powerful deodorizer, having a number of attractive features. For example, ozone is pervasive in a closed space. Ozone is also highly effective as a viricide, and is inexpensive to administer, as ozone generators are plentiful and easy to install and operate.

Ozone is naturally formed, particularly in the upper atmosphere, when high-energy ultraviolet rays sever conventional oxygen (O₂) bonds, creating free radical oxygen atoms, which then react with other O₂ molecules to form ozone (O₃). Ozone is also formed naturally during lightning storms, at ocean beaches and waterfalls.

The structure of ozone is highly reactive, and consequently ozone has a short half-life (about 30 minutes). When ozone breaks down, it produces oxygen and a free radical oxygen atom. This oxygen free radical is a powerful oxidant.

There are several ozone generators described in the prior art. For example, U.S. Pat. No. 5,904,901 to Shimono discloses a deodorization/odor-removal/disinfection method and deodorization/odor-removal/disinfection apparatus.

Prior art relating to the sterilization of hotel rooms and the like using ozone includes JP4038957A2, which discloses a determination of the time a room should be exposed to a particular concentration of ozone. JP2237565A2 discloses an indoor sterilizing method which includes placing an ozone generator in a room, generating a level of ozone, leaving the ozone at that level for a period of time, and then decomposing the ozone.

What is missing in the prior art is a consideration of other factors besides ozone concentration and time needed to use the ozone effectively as a sterilizing agent. Also while ozone is recognized as having sterilizing properties, few tests have been carried out to determine its efficacy on new diseases.

### BRIEF SUMMARY OF THE INVENTION

A method of sterilizing a closed environment is provided, including (a) placing a ozone generator into said closed environment; (b) generating ozone to a predetermined ozone concentration; (c) increasing the humidity of said closed environment; (d) maintaining said predetermined ozone concentration for a predetermined period of time; (e) after the expiry of said period of time, depleting said ozone; and (f) when said ozone concentration is reduced to a predetermined safe level, signalling.

An ozone generator is provided including a humidifier; a timer; ozone generation means; ozone depletion means; movement means; signalling means; and detection means for detecting ozone concentration and humidity of a close environment.

A method of inactivating a quantity of Norwalk virus in a closed environment is provided, comprising exposing the closed environment to an ozone concentration of 20 to 35 ppm for 30 to 70 minutes. It is beneficial to elevate the humidity of said closed environment while exposing the closed environment to said ozone concentration.

### BRIEF DESCRIPTION OF THE DRAWINGS

FIG. 1 is a perspective view of an ozone generator according to the invention;

FIG. 2 is a block diagram thereof; and

FIG. 3 is a flow chart showing the use of an ozone generator according to the method.

### DETAILED DESCRIPTION OF THE INVENTION

A difficulty with using ozone as a disinfectant is that the concentrations and exposure times required for ozone to be an effective disinfectant are considered to be toxic for humans. Such concentrations and exposure times may also generate noxious by-products from chemical reactions with fabrics commonly found indoors particularly in carpets). For example, ozone may react with chemicals in carpets to create formic acid. Exposure to elevated ozone concentrations may irritate the lungs and have other side effects, including throat irritation, shortness of breath and coughing. Consequently several agencies have discouraged the use of ozone to sanitize indoor spaces and have set maximum safe levels of ozone to be from 0.05 parts per million (“ppm”) to 0.10 ppm for an eight hour exposure. Unless otherwise stated to references to ozone in this document refer to ozone in a gaseous state.

Ozone is effective against many types of organisms, including retroviruses, both enveloped and naked viruses, bacteria and fungus. Specific diseases which ozone has been shown to be effective against include: MS2 Coliphage; Poliovirus Type 1 and Type 3; Hepatitis A; Enteroviruses; Rotaviruses; HIV; SA11 and enteric viruses; Influenza viruses; the Norwalk virus and Rhinoviruses. Ozone may also be used to kill SARS viruses, infectious prions, and bacteria, and can also decontaminate foodstuffs and sterilize medical equipment. For further information about the general efficacy of ozone as a viricide see Appendix A entitled “Ozone: A Viricidal Agent for Conventional and Emerging Viruses”.

The level of ozone concentration required to be effective and achieve over 95% (and often over 99%) morbidity rates of viruses and other disease causing agents varies depending on the time the agents are exposed to the ozone. One constant is that the ozone concentration is well above the safe levels for human exposure and therefore precautions should be taken to prevent such exposure. Ozone concentrations of approximately 100 ppm are very effective to kill infectious agents and may require exposure times for as little as 10 to 15 minutes. Lower ozone concentrations (for example as low as 20 ppm to 25 ppm) are also effective, although, in the case of such lower quantities of ozone, it takes more time (such as 20 to 30 minutes) for the ozone to be effective.

### The Process of Using Ozone as a Disinfectant.

The present invention includes portable equipment, specifications and operating procedures to provide adequate ozone exposure in indoor spaces to achieve an effective degree of sanitization followed by rapid removal of the ozone and attendant gaseous by-products from the reaction of ozone with carpet and furniture fabrics.

The invention includes identifying the variables impacting the safe and effective use of ozone as a disinfectant in the hospitality and other industries. In summary, the invention provides for:
1. Rapid elevation of ozone levels within a fixed interior space, combined with suitable humidity control and turbulent airflow;
2. Measurement and control of effective exposure to sanitizing ozone optimum for the use of ozone as a viricide for use on various surfaces commonly found in the fixed space (e.g. in a hotel room); and
3. Rapid consumption of ozone and gaseous aldehyde by-products to reduce their concentrations to levels deemed safe for human exposure.

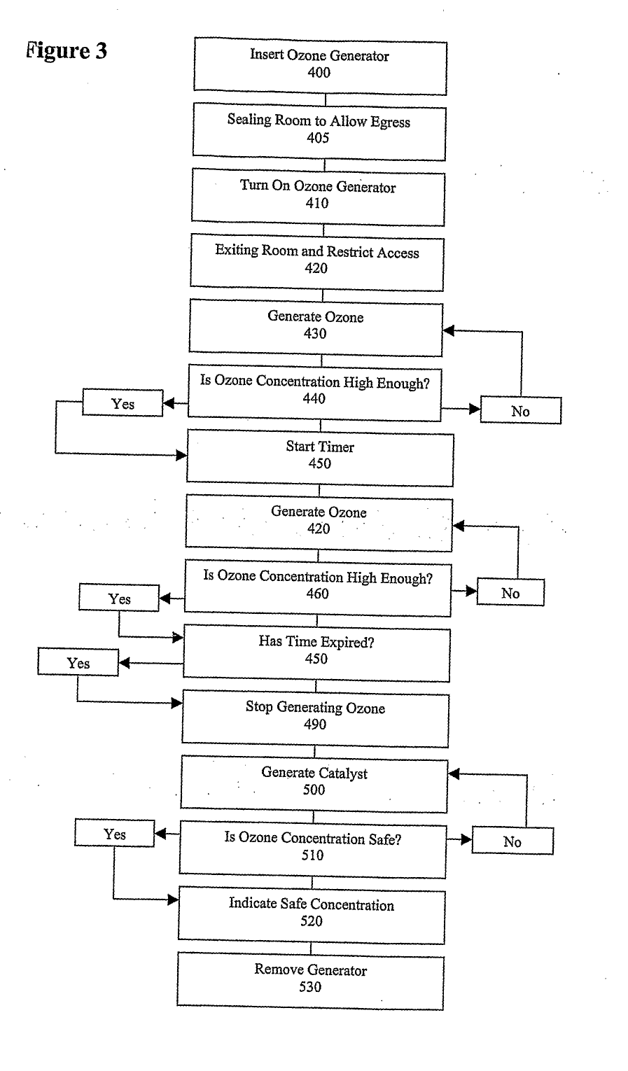

As an example, as seen in FIG. 3, a preferred method according to the invention may include the following steps:

a) inserting a portable ozone generator in a closed interior environment, such as a hotel room (step 400);

b) elevating and maintaining the ozone concentration in the closed environment to a level sufficient to act as a disinfectant and viricide taking into account the humidity, size, temperature and airflow of the closed environment (step 410);

c) restricting access to the closed environment while the ozone levels are elevated to prevent human exposure while the ozone concentration is dangerously high (step 420);

d) consuming the ozone and any gaseous aldehyde by-products (possibly including the use of a catalyst) for a period of time taking into account the ozone levels, the humidity, the temperature, the airflow and the size of the closed environment, until the ozone concentration is below toxic levels (step 500); and

e) removing the portable ozone generator from the closed environment (step 530).

In further detail, with reference to FIG. 3, the process begins with the insertion of an ozone generator into a closed interior room (step 400). Examples of appropriate rooms include hotel rooms, cruise ship cabins, hospital rooms and airplane cabins. The room is preferably easily cut off from public access (step 405) so that employees or guests will not be exposed to high concentrations of ozone. Examples of closing a room include simply locking the door of a hotel room or cruise ship cabin when it is not in use by a guest. Windows should be closed and any ventilation systems turned off. Note that as the user is still inside the room, it is important that it not be difficult to exit the closed environment quickly.

The user will then preferably turn on the ozone generator (step 410) and exit the closed environment (step 420). Preferably the ozone generator has a timer such that when it is turned on, there is a period of time (for example one or two minutes) before the ozone generator will begin generating ozone. This provides time for the user to exit the closed environment without exposure to the ozone.

In some embodiments of the invention, the user will have to adjust the ozone generator so that it will produce the appropriate amount of ozone within the appropriate time based on humidity, temperature, air flow and the like. It may also be necessary for the user to enter information about the room size (for example a menu of options such as “Suite”, “Single” or “Double” could be displayed from which the appropriate selection is made). Alternatively, in a preferred embodiment, the ozone generator will measure these indicia, like temperature and humidity and automatically calculate the appropriate concentration of ozone and time that it should be maintained.

The next step is to restrict access to the closed environment (step 420) while the ozone concentration is elevated to prevent exposure to the ozone. The closed environment does not need to be airtight, for example closing the doors and windows of a hotel room is sufficient. Fans within the room should be turned off. The entrance to the closed room should be locked and possibly a sign or warning light used to indicate that entry should not be permitted during the period when ozone concentrations are elevated.

The ozone generator may also be able to adjust certain factors of the closed environment in order that the ozone will more efficiently act as a viricide. For example the ozone generator may also have the ability to increase the humidity of the closed environment, which make the ozone more efficient as a viricide. This in turn may allow the ozone generation and ozone concentration maintenance periods to be shorter.

The ozone generator then generates ozone (step 430) until the appropriate concentration is reached (step 440). This concentration is maintained (step 440) for the specified period of time (steps 460 and 470). Examples of sufficient ozone concentrations in a typical hotel room or cruise ship cabin would be 40 to 50 ppm for about 10 to 15 minutes or a concentration between 20 and 35 ppm for about 20 to 35 minutes. Increased humidity levels can shorten the time needed.

After the ozone concentration has reached the desired level and has been maintained at that level for a sufficient period of time (step 480), the ozone generator stops generating ozone (step 490). The ozone then begins to dissipate, both naturally, and preferably by the generation of an appropriate catalyst (step 500). The ozone concentration is measured (step 510) as the ozone is dissipated (as are the gaseous aldehyde by-products) for a period of time taking into account the ozone levels, the humidity, the temperature, the airflow and the size of the closed environment, until the ozone concentration is below toxic levels at which point the ozone generator signals the room is safe to enter using an LED, a noise or the like (step 520).

Once the appropriate amount of time has passed and the ozone generator has indicated the ozone concentration is sufficiently low, the ozone generator is removed from the closed environment and can be used in another closed environment (step 530).

### The Ozone Generator

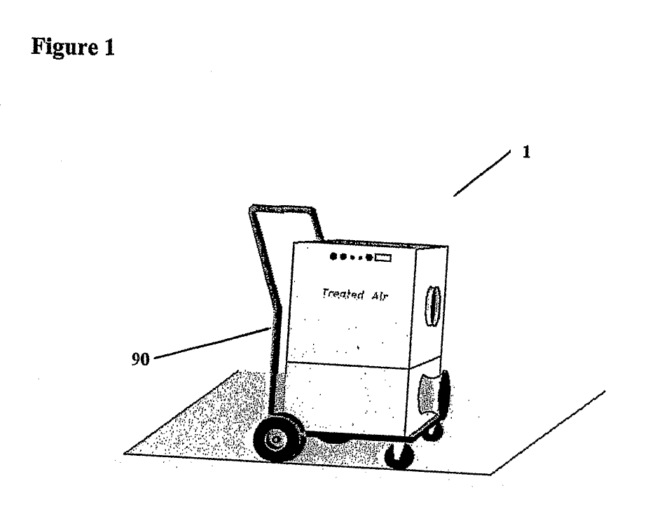

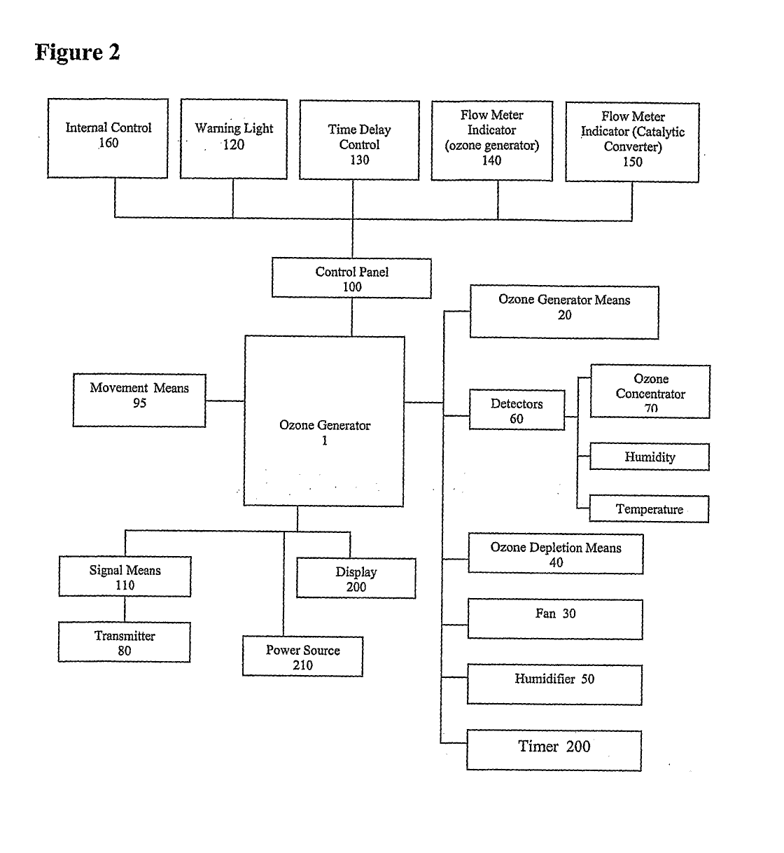

The previously described method can be used with a variety of zone generators, however a preferred ozone generator is shown in FIGS. 1 and 2. The ozone generator, generally indicated as 1, preferably generates gaseous ozone using corona discharge or ultra violet light or other ozone generation means 20 as known in the art. The corona discharge process can create ozone using air in the closed environment passed through ozone generator 1 by fan 30, or alternatively air can be introduced into the closed space or industrial or medical oxygen. The ozone generator preferably also has an ozone depletion means 40 such as an ozone scrubber or catalytic converter, and a humidifier 50. Also the generator preferably has detectors 60, particularly a detector for the concentration of ozone 70 in the closed environment.

The ozone scrubber or catalytic converter (also referred to as ozone depletion means) allows the ozone generator 1 to quickly deplete the concentration of ozone to levels which are acceptable for human habitation. A catalytic converter uses substances such as manganese dioxide, treated or activated carbon, or a combination of both. A catalytic converter will also deplete the ozonated air of aldehyde, nitroxides and any other noxious gases generated as by products of the ozone reacting with articles in the environment, such as carpets. Activated carbon can be used to reduce the levels of noxious by-products caused by the ozone reactions with carpet and the like. Another factor in the depletion of the ozone, is the natural half-life of ozone, which is about 25 to 30 minutes.

The ozone generator 1 also preferably has a humidifier 50. The humidifier 50 is used to modify the relative humidity of the air volume in conjunction with the other operations of the generator. Accordingly, the humidifier may be used before, during and/or after the ozone generation process as necessary. As higher levels of humidity tend to make the ozone more effective as a viricide, in most environments the humidifier 50 will be engaged to increase the humidity of the closed environment.

The ozone generator 1 should either be sufficiently small and light enough to be easily carried or should be mounted on a trolley 90 (as seen in FIG. 1) or affixed with other movement means 95, such as wheels. Alternatively the ozone generator 1 could be a fixture with the closed environment. In a preferred embodiment the generator is affixed to an ergonomically suitable trolly 95 so that it can easily be moved from room to room within a larger structure (such as a hotel).

The ozone generator 1 also preferably has detectors 60 means to detect the ozone levels 70 within the closed environment. This is used so that users can determine when the ozone concentration is low enough to allow safe entry into a room. In a preferred embodiment of the invention the generator will indicate that the ozone concentration is safe and transmit a signal using transmitter 80 to a device (a mobile phone, PDA or the like) indicating that the room is now safe to enter. Alternatively the signal can be sent to a control panel 100 which will manipulate a LED on the outside of the room (e.g. red for high concentrations, and green for lower safe concentrations).

In yet another alternative embodiment, the generator has an LED or similar signal emitting means 110 such that a user entering the closed environment will be immediately aware that the ozone levels are still too high for safety and can exit the environment.

The ozone generator also preferably has one or more of the following components:

1. a timer 110 to record the number of hours or minutes the generator has been operating and to turn off the generator when the appropriate time has passed;
2. a warning light 120 to indicate that the ozone generator is generating ozone;
3. a time delay switch 130 to allow for a delay before the ozone generator beings to generate ozone, allowing the user to exit the closed environment;
4. one or more other time delay switches for the operation of the scrubber, humidifier, and other features;
5. a flow meter 140 to indicate that there is an air flow moving through the ozone generator;
6. a flow meter 150 to indicate that there is an air flow moving through the catalytic converter;
7. an instrument panel to indicate which part of the apparatus is working either individually or with others;
8. further alarms included in the instrumentation that would indicate a malfunction of the generator;
9. an internal control 160 to allow for variance of the ozone concentration to be achieved;
10. sliding inspection panels to allow for easy maintenance and inspection of the apparatus; and
11. separate electric fittings and plugs to allow for ancillary apparatus such as an additional scrubber to be connected to the apparatus.

Ozone generator 1 also has power source 210 which can be a plug for insertion into a suitable outlet, or batteries. Ozone generator also has displays 200 preferably showing the current ozone concentration, humidity and temperature.

### USE EXAMPLE 1

### Hotels

Hotels are used to frequent visitors in a particular room, often only staying a single night. Hotels are also one of the worst effected by disease scares as in the case of SARS, as tourism is one the industries most keenly effected. Hotels have also been using ozone at low concentrations to reduce odours in rooms.

As used in hotels according to the method, a maid after initially cleaning a vacated room (preferably after the guest had checked out) would place the ozone generator in the room set it for the specified ozone concentration and time, and leave the room (and locking the door), returning when the time had passed and the ozone concentration was reduced to safe levels. The ozone generator can then be taken to the next appropriate room.

At the end of the process, the ozone would kill the viruses, bacteria and fungi left by the departing person(s).

### USE EXAMPLE 2

### Airplanes

The airline industry is another industry prone to losses when fear of a disease outbreak strikes. To use the method according to the invention on an airliner, after the airliner is initially cleaned, one or more ozone generators should be turned on and the selected ozone concentration maintained for a period of time. During this time access to the interior of the airplane should be prevented.

Once the necessary time has passed, and the ozone concentrations are safe, the interior of the airplane is access and the ozone generators can be removed.

### USE EXAMPLE 3

### Cruise Ships

Cruise ships present an environment where a disease can spread rapidly due to the confinement of a large number of people in a small environment. The method according to the invention is useful when the ship is docked and few are about, in which case it is used in a manner very similar to that of the hotel example described previously. Alternatively, the ozone generator could be used within a room when the inhabitants report certain symptoms.

### USE EXAMPLE 4

### Hospitals

A yet further example of a location in which to use the method according to the invention is a hospital. Obviously hospitals are areas in which viruses, bacteria and other disease causing agents are common as those diseased may end up in such a location. When a hospital room is vacated, perhaps even only temporarily, the method according to the invention could be carried out to kill any viruses or bacteria left by the last patient staying in such rooms. It may be beneficial to use the ozone generator in emergency areas and the like when such area is exposed to a particularly problematic disease (such as SARS).

### Effectiveness of Ozone

Generally tests were conducted to show that ozone gas can efficiently inactivate (kill) five selected viruses tested, namely, herpes simplex virus, influenza virus, corona virus, poliovirus and rhinovirus. These viruses were found to be vulnerable to ozone in a gaseous state on surfaces such as glass, plastic, steel, wood and fabric. Increasing the concentration of ozone and greater times of exposure were more effective, as anticipated, and increasing the relative humidity also significantly increased the antiviral efficacy.

### Experiment #1

Ozone was generated within a chamber to provide an ozone concentration of approximately 100 ppm for 30 minutes on a variety of surfaces, including glass slides, steel disks, etc., Relative humidity and temperature were recorded.

Herpes Simplex Virus (“HSV”), Feline calicivirus (“FCV”), and Mulluscum Contagiosum Virus (“MCV”) were all dramatically inactivated by exposure to ozone gas. Typically a dosage of 100 ppm for 20-30 minutes reduced the virus by more than 99%. Shorter exposure times resulted in significant though smaller reductions. Thus 10 minutes inactivated approximately 90-95% virus infectivity, whereas shorter time periods were less effective. It appeared, from a number of the time course studies made, that a period of between 5 and 10 minutes exposure to ozone was required to absorb the gas and effect the appropriate chemical processes, before loss of infectivity occurred. Poliovirus was also inactivated by ozone under similar conditions.

Exposure of the viruses to ozone was made on samples dried on six different surfaces, relevant to materials encountered in the hospitality industry, glass, plastic, stainless steel, wood, fabric, and carpet. Several viruses were evaluated on each surface, though not every permutation was feasible because of time constraints and cost. In general, the viruses were susceptible to ozone on glass, plastic, steel, wood, and fabric.

The results of numerous time course experiments, with different virus-surface combinations, confirmed that increasing time of exposure resulted in greater inactivation of virus, and in some cases no vilus infectivity could be detected at all after 30 minutes exposure.

In several experiments the effect of relative humidity was examined by incorporating a container of warm water into the chamber during exposure. It was difficult to control exact humidity levels in this manner; nevertheless it was clear that in high humidity virus was inactivated by ozone much more efficiently than in ambient humidity (which was usually 45-50%).

### Experiment #2

A further experiment was conducted to test the effect of ozone gas against selected viruses, under conditions similar to those in a hotel room. The aim was to measure the amount of ozone inactivation of HSV in several different locations within a test room and to compare the efficacy of ozone inactivation of three different viruses (HSV, poliovirus and rhinovirus) placed within the test room.

The three samples of HSV were inactivated (killed) by 98%, 99.4% and 97.8%. The ozone concentration was 28 ppm and the time of exposure was 60 minutes (it also took 30 minutes to reach that ozone concentration from a starting point of 0).

As the inactivation was similar at three different locations within the room indicating that the ozone gas should be very effective at inactivating viruses within a large room.

### Experiment #3

A further experiment was conducted to evaluate the effect of ozone gas against FCV, the surrogate virus for Norwalk virus, in comparison with HSV and poliovirus, under conditions of reduced ozone doses and high humidity.

The FCV was inactivated by 99.91%; the poliovirus was inactivated by much more than 99.6%; and the HSV was inactivated by much more than 99%. The closed interior environment used for these tests, was provided an atmosphere of high humidity, and with substantially reduced ozone dosage (between 20 ppm and 40 ppm) for about 15 minutes. It was concluded that FCV can be inactivated more than 99.9% by exposure to ozone gas in the presence of high relative humidity and it should be possible to inactivate this virus (and by extrapolation Norwalk virus) even further by optimizing the ozone dosage and humidity.

### Experiment #4

A further experiment was conducted to develop an appropriate and relevant experimental system for testing the efficacy of quantified ozone doses in inactivating (i.e. killing) known amounts of several important human viruses; to derive viricidal killing curves for known doses of ozone gas against samples of dried viruses on several different surfaces relevant to the hospitality industry; to compare the viricidal efficacy of ozone gas against five selected viruses known to be important in human health; to examine the effects of different parameters on the viricidal efficacy of ozone gas, including: concentration of ozone, time of exposure, and relative humidity; and to consider the potential for additional applications of ozone gas as a sterilizing agent in other situations where viral and microbial agents could pose threats.

The experiments showed that ozone gas can efficiently inactivate (kill) all of the five selected viruses tested, namely, herpes simplex virus, influenza virus, corona virus, rhinovirus, and poliovirus. These viruses are vulnerable to ozone gas in the dried state on different surfaces, such as glass, plastic, steel, wood and fabric. Increasing doses of ozone and greater times of exposure were more effective, as anticipated, and increasing relative humidity also significantly increased the antiviral efficacy.

Based on these results we conclude that the viruses tested are efficiently inactivated by gaseous ozone, on each of the surfaces tested, under conditions relevant to practical applications. Therefore ozone gas also has potential as a safe antiviral and anti-microbial agent in various other situations that are accessible to a small, portable, ozone generating machine.

HSV, FV, and MCV were all dramatically inactivated by exposure to ozone gas. Typically a dosage of 100 ppm for 20 to 30 minutes reduced the virus by more than 99%. Shorter exposure times resulted in significant though smaller reductions. Thus 10 minutes inactivated approximately 90-95% of the virus infectivity, whereas shorter time periods were less effective. It appeared, from a number of the time course studies made, that a period of between 5 and 10 minutes exposure to ozone was required to absorb the gas and effect the appropriate chemical processes, before loss of infectivity occurred. Presumably oxidation of particular viral components is required, and that this process requires several minutes. Following this process, inactivation, i.e. loss of infectivity, is rapid.

Exposure of the viruses to ozone was made on samples dried on six different surfaces, relevant to materials encountered in the hospitality industry, glass, plastic, stainless steel, wood, fabric, and carpet. Several viruses were evaluated on each surface. In general, the viruses were susceptible to ozone on such surfaces.

In several experiments the effect of relative humidity was examined by incorporating a container of warm water into the chamber during exposure. It was difficult to control exact humidity levels in this manner; nevertheless it was clear that in high humidity the virus was inactivated by ozone much more efficiently than in ambient humidity (which was usually 45-50%).

### Experiment #5

Further experiments were conducted to determine the inactivation of the Norwalk virus and to do research regarding an ozone scrubber. It had already been demonstrated that several viruses, including the feline calicivirus (the recommended surrogate virus for testing Norwalk virus susceptibility to anti-viral agents), could be inactivated by ozone gas.

The objective of the experiment was to optimize the ozonation protocols in order to minimize the effective dose and exposure times required, to determine the degree of relative humidity preferred, and to confirm the optimal protocols for virus specimens resembling field conditions (i.e. in different biological fluids and on “unclean surfaces”).

The feline calicivirus used in these test procedures because Norwalk virus itself cannot be grown and measured in cell cultures. However, once optimal conditions for ozone inactivation of calicivirus have been determined, then reference stool specimens known to contain Norwalk virus can be tested.

The data confirmed that FCV, and therefore Norwalk virus, can be efficiently inactivated by our ozone generator under standard conditions and at durations, temperature and humidity levels which would be appropriate for the cruise liner and hotel industries.

Other disease causing agents such as viruses and bacteria that ozone is effective against include: Clostridium difficile (a human pathogenic bacterium of the gut); Antibiotic-Resistant bacteria (E. coli, Staphylococcus and Streptococcus, including the multiple antibiotic—resistant strain (MRSA) of Staph); Candida albicans (a yeast); and fungi growing on different surfaces.

Although the particular preferred embodiments of the invention have been disclosed in detail for illustrative purposes, it will be recognized that variations or modifications of the disclosed apparatus lie within the scope of the present invention.

### APPENDIX A

### Ozone:

### A Virucidal Agent For Conventional and Emerging Viruses

TABLE OF CONTENTS

| 1.0 | INTRODUCTION | | 2 |
|---|---|---|---|
| | 1.1 | The Assignment | 2 |
| | 1.2 | Scope of the Research and Analysis Report | 2 |
| 2.0 | ANTIPATHOGENICITY OF OZONE | | 2 |
| | 2.1 | Ozone - Overview | 2 |
| | 2.2 | Viruses - Brief Overview | 4 |
| | 2.3 | Ozone as a Disinfectant - Mechanisms of Action | 4 |
| | 2.4 | Side Effects and limitations of Ozone Use | 5 |
| 3.0 | CONVENTIONAL & EMERGING VIRUSES OF CLINICAL IMPORTANCE | | 7 |
| | 3.1 | Conventional Viruses | 7 |

| | | |
|---|---|---|
| 3.1.1 | Enteroviruses | 7 |
| 3.1.2 | Gastroenteritis | 9 |
| 3.1.3 | Influenza | 10 |
| 3.1.4 | Viral Respiratory Infections | 11 |
| 3.1.5 | Other Airborne Viruses | 13 |

| | 3.2 | Emerging Viruses | 14 |
|---|---|---|---|
| | 3.3 | Virucidal Activity of Ozone - Historical Perspective | 15 |
| | 3.4 | Virucidal Activity of Ozone - Prospective View | 21 |
| 4.0 | PILOT MARKET ANALYSIS | | 22 |
| | 4.1 | Airline Industry | 23 |
| | 4.2 | Hospitality Industry | 24 |
| | 4.3 | Hospitals, Nursing Homes, Daycares, and Laboratories | 25 |
| | 4.4 | Sanitization of Industrial Fabric | 26 |
| | 4.5 | Public Washrooms, Physician's Waiting Rooms, Gyms and Locker | 26 |
| | | Rooms, Airports and Tourist Areas | |
| | 4.6 | Medical Equipment and Medicine | 26 |
| | 4.7 | Granaries, Sawmills, and Scientific Research involving Rodents | 26 |
| | 4.8 | Other Applications | 27 |

| | | |
|---|---|---|
| 5.0 | CONCLUSIONS & RECOMMENDATIONS | 27 |
| 6.0 | DISCLAIMERS AND LIMITATIONS | 29 |
| 7.0 | BIOSTAR MANAGEMENT INCORPORATED | 30 |
| 8.0 | REFERENCES | 30 |

### 1.0 INTRODUCTION

### 1.1 The Assignment

The quality of indoor air in areas such as aircraft (and other public transportation systems), hospitals, offices and other enclosed spaces is a significant occupational health concern. And it is not only staff and workers whose health is at stake, but also the customers, frequent flyers, office workers, and hospital patients are all threatened by poor air quality. Treated Air Systems Manufacturing, Inc. (TASM) is a British Columbia-based company that manufactures ozone generators. Ozone is a powerful oxidant capable of removing odours, neutralizing toxic gases, decontaminating water and disinfecting pathogens. TASM has retained BioStar Management, Inc. (BioStar) to prepare a Research and Analysis Report (the Report) on the efficacy and potential use of ozone as a virucidal (virus inactivating) agent.

### 1.2 Scope of the Research and Analysis Report

The recent outbreak and spread of global diseases (such as Severe Acute Respiratory Syndrome, SARS) are worrying. The increasing frequency with which these outbreaks are occurring is a trend that is likely to continue, presenting growing concerns about emerging viral diseases and the persistence of conventional viruses of clinical importance. In response to concerns such as these, TASM is interested in expanding its potential market to include utilization of ozone as a virucidal agent in the hospitality, aircraft, medical and other industries. Accordingly, to facilitate this business opportunity, the scope of this Report includes research and analysis of anecdotal evidence and other scientific information in the public domain pertaining to the efficacy of ozone as a virucidal agent.

The specific goals of the Research & Analysis Report were to:
1. Research, review, synthesize and integrate relevant information available from the anecdotal studies published and other relevant information in the public domain pertaining to ozone as an anti viral agent
2. Prepare a clear, concise report and analysis based on the research and data collected.

Public domains such as NIH, Medline, Pubmed, CHR, Recap, Google and other databases were searched for relevant published articles, relevant Ozone technologies and other related information. The data of the publications are reported in a table or/and in discussion format.

### 2.0 ANTIPATHOGENICITY OF OZONE

### 2.1 Ozone—Overview

Ozone is formed in the upper atmosphere (the troposphere) when high energy ultraviolet (UV) rays sever conventional oxygen (O₂) bonds, creating free radical oxygen atoms, which then react with other O₂ molecules to form ozone. Ozone can also be formed during lightning storms, and at ocean beaches and waterfalls. It has been widely used in medical applications for almost 100 years. Recent technological advances have made it a cost-effective air purification agent.

Ozone (O3) is a classic example of a resonance structure. A double-bond resonates between a central oxygen molecule and one of two other oxygen molecules. FIG. 2.1 illustrates ozone's resonance structure.

The resonating structure of ozone is highly reactive, and consequently ozone has a short half-life. When ozone breaks down, it produces oxygen and a free radical oxygen atom (a negatively charged single oxygen atom). This oxygen free radical is a powerful oxidant. In the presence of UV light that reaches the troposphere, the oxygen free radical can combine with ozone to form two O₂ molecules. It is this process that allows ozone to absorb much of the UV light (a DNA-damaging agent and a mutagen) that would otherwise penetrate the Earth's atmosphere. Generation of ozone in the troposphere and absorption of UV light by ozone is depicted in FIG. 2.2.

As an oxidant and a generator of oxygen, ozone has five key benefits:
Oxidization of nuisance odors: tobacco smoke, food odors, body odors, urine, pet odors, chemical odors, etc
Control of airborne micro-particulates: dust, smoke, lint, fibers, etc.
Sterilization of micro-organisms: bacteria, viruses, fungus, moulds, mildew, germs, etc.
Slower spoilage rates/increased shelf life: fruits, meats, vegetables, fish, cut flowers, etc.
Faster growth rates: plants, flowers, poultry, pigs, etc. by providing a cleaner healthier environment.

### 2.2 Viruses—Brief Overview

This section provides a brief overview of viruses with a focus on conventional and emerging viruses of clinical importance, as well as viruses that may be amenable to management through ozone inactivation. This overview also serves as a reference for viruses and technical terms used in subsequent sections.

Viruses are obligate intracellular parasites. They lack the biochemical machinery (organelles and enzymes) that are required to thrive outside living cells. They are relatively basic in composition with a nucleocapsid that is collectively composed of a genome (DNA or RNA) and proteins. Viruses are generally classified as “enveloped” or “non-enveloped” (or “naked”) viruses. In enveloped viruses, the nucleocapsid is surrounded by a lipid membrane, which is acquired during assembly of the viruses from infected cells. This lipid envelope contains moieties, such as glycoproteins (modified proteins), that facilitate virus attachment and entry during subsequent cycles of infections. Non-enveloped or naked viruses do not have this lipid membrane. Instead, the nucleocapsid core is surrounded by other structural proteins that are exquisitely organized into a spore-like structure.

### 2.3 Ozone as a Disinfectant—Mechanisms of Action

Ozone's disinfectant properties have been known since the 19th century. The first ozone disinfection experiment took place in 1886 (Shnonbein, 1886). More recently, the use of ozone as an environmental decontaminant has begun to be explored, and has been demonstrated in several studies.

Because ozone will spontaneously degrade to produce a free radical oxygen atom, its oxidizing power is very strong. It is this oxidizing power that is the source of its antiviral, bactericidal and fungicidal properties. The table below summarizes the nature of ozone's disinfectant properties.

TABLE 2.1
A summary of the disinfectant properties of ozone.

| Type of Organism | Method of Inhibition | Scope of Applicability | References |
|---|---|---|---|
| Enveloped Viruses | Oxidation of lipid | All viruses with lipid | Sunnen, 1994 |
| | glycoprotein membrane | glycoprotein membrane | |
| “Naked” Viruses | Cleavage of nuclear | Less effective against | Sunnen, 1994 |
| | material | naked viruses | |
| | | compared to enveloped | |
| | | viruses | |
| Bacteria | Oxidation of lipid | Gram-positive and | Ishizaki et al., 1987 |
| | glycoprotein membrane | gram-negative bacteria | |
| Fungus | Mechanism poorly | Inhibit budding cells; | Matus et al., 1981; |
| | understood | may actually stimulate | Matus et al., 1982 |
| | | growth at low levels | |

Several mechanisms have been proposed to explain ozone's antiviral properties. The simplest mechanism proposed is the direct oxidation of the lipids in the envelope that surrounds the viruses. The free radical oxygen generated by the degradation of ozone can cause a “chain reaction” within the lipid brayer of viruses. That is, the free radical partially oxidizes a lipid molecule, which then generates another free radical, which in turn oxidizes another lipid, creating a “Domino Effect”. Most viruses that possess a lipid glycoprotein envelope, including retrovirus, hepatitis B and C, and Herpes Type 1 and 2, are susceptible to ozone due to rapid and cascading oxidation unsaturated fatty acids that make up the lipid bilayer. As noted above, non-enveloped or “naked” viruses, lack a lipid envelope. In these organisms, ozone diffuses across the protein capsid (coat) that encloses the nuclear material, and cleaves the DNA or RNA, resulting in viral inactivation.

In addition to virucidal properties, ozone is also bactericidal and fungicidal. Ozone has been shown to destroy bacteria such as Escherichia coli (E. coli), staphylococcus, and other bacterial pathogens. The mechanism through which ozone inactivates bacteria is similar to the mechanism by which it destroys viruses. When O₃ molecules come in contact with the bacterial membrane, it rapidly oxidizes the lipids, leaving as a product an oxidized lipid and a free-radical lipid. The free-radical lipid subsequently oxidizes another lipid, and so on (Ishizaki et al., 1987). Ozone's fungicidal mechanism is poorly understood, however it appears that ozone is most effective in inhibiting “budding” cells (cells that are dividing), while fungal cells at other stages are less inhibited by ozone (Matus et al., 1981). Some studies have shown that low levels of ozone may actually stimulate fungal growth, while higher levels inhibit growth Matus et al., 1982.

### 2.4 Side Effects and Limitations of Ozone Use

The use of ozone as an antiviral agent is limited due to its potential to cause adverse side effects, and thus its use in inhabited areas is highly restricted. Ozone should never be utilized to eliminate the risk of transmission of pathogens in indoor environments that are occupied by humans, pets or other animals, for the reasons considered below.

Ozone poses a health risk, and can have several adverse effects. For example, ozone has the ability to cause irritation to lungs when inhaled (www.epa.gov/iedweb00/pubs/ozonegen). Relatively low amounts can cause chest pain, coughing, shortness of breath, and throat irritation. Ozone may also worsen chronic respiratory diseases such as asthma and compromise the ability of the body to fight respiratory infections. People vary widely in their susceptibility to ozone. Healthy people, as well as those with respiratory difficulty, can experience breathing problems when exposed to ozone. Health Canada has issued warnings on the use of the direct and purposeful generation of ozone in indoor occupied spaces (www.hc-sc.gc.ca/english/protection/warnings/1999/99_{—}62e.html.). Additionally, following a review of current information and in consultation with Health Canada and others, the Canadian Standards Association (CSA) recently made the decision not to certify ozone generators for household use and issued new interim requirements for commercial units. Health Canada advises owners of commercial ozone generators to discontinue use in indoor occupied space.

Several US federal agencies have established health standards or recommendations to limit human exposure to ozone. These exposure limits are summarized in the table below (www.epa.gov/iedweb00/pubs/ozonegen).

### Ozone Heath Effects and Standards

| Health Effects | Risk Factors | Health Standards |
|---|---|---|
| Decreases in lung function | Increase in ozone air | The Food and Drug |
| | concentration | Administration (FDA) requires |
| | | ozone output of indoor medical |
| | | devices to be no more than 0.05 ppm. |
| Aggravation of asthma | Greater duration of exposure for | The Occupational Safety and |
| | some health effects | Health Administration (OSHA) |
| | | requires that workers not be |
| | | exposed to an average |
| | | concentration of more than 0.10 ppm |
| | | for 8 hours. |
| Throat irritation and cough | Activities that raise the breathing | The National Institute of |
| | rate (e.g., exercise) | Occupational Safety and Health. |
| | | (NIOSH) recommends an upper |
| | | limit of 0.10 ppm, not to be |
| | | exceeded at any time. |
| Chest pain and shortness of | Certain pre-existing lung | The Environmental Protection |
| breath | diseases (e.g., asthma) | Agency (EPA)'s National |
| | | Ambient Air Quality Standard for |
| | | ozone is a maximum 8 hour |
| | | average outdoor concentration of |
| | | 0.08 ppm. |
| Inflammation of lung tissue | | |
| Higher susceptibility to | | |
| respiratory infection | | |

(* ppm = parts per million)

Although a number of studies indicate that ozone may reduce airborne concentrations and inhibit the growth of some pathogens, the ozone concentrations required to achieve significant pathogen inhibition are roughly 5-10 times higher than public health standards. Furthermore, even at higher levels, ozone may have no effect on biological contaminants embedded in porous material, such as carpeting. In other words, ozone produced by ozone generators may inhibit the growth of some biological agents while it is present, but it is unlikely to fully decontaminate the air unless concentrations are high enough to be a health concern if people are present.

In addition to the harmful effects of ozone itself, ozone can react with many common indoor substances to form harmful by-products (www.epa.gov/iedweb00/pubs/ozonegen). For example, in carpets, especially new, ozone can reduce many of the chemicals present to form a variety of aldehydes. Ozone is also known increase indoor concentrations of formic acid. Both aldehydes and formic acid are lung irritants. Some of the potential by-products produced by ozone's reactions with other chemicals are themselves very reactive and capable of producing irritating and corrosive by-products. Given the complexity of the chemical reactions that occur, additional research is needed to more completely understand the complex interactions of indoor chemicals in the presence of ozone (Fan et al., 2003).

### 3.0. CONVENTIONAL AND EMERGING VIRUSES OF CLINICAL IMPORTANCE

Conventional viruses are ones that have evolved with the host, persist in the population and give rise to clinical symptoms under defined conditions. Emerging viruses are those that have newly appeared (through mutations) or already exist in nature and cause manifestations of widespread diseases mostly due to changes in the ecosystem, demographics and climate. In general communicable disease are operationally defined by their modes of transmission with the four main transmission categories being:
Food and waterborne enteric transmission
Airborne respiratory transmission
Sexual transmission via direct contact
Vector (animals, insects, etc) and blood borne transmission

For its potential use as a virucidal agent through fumigation for decontamination of space and objects, it is appropriate to address viruses that are transmitted through the first two modes listed: food and waterborne enteric transmission and airborne respiratory transmission Viruses that are transmitted by sexual contact or those that are vector or blood borne may be amenable to treatment with ozone but they are beyond the scope of this Report.

### 3.1 Conventional Viruses and Disease

Prior to discussion of these viruses, it is important to note that there is a marked difference in the manner in which viruses are classified between disciplines. In General Virology, virus families are grouped based on genetic relatedness. In contrast, clinical (medical) virology classifies viruses based on the diseases that they cause. For the purposes of this report, viruses will be described based on their clinical (medical classification) so that viruses that are amenable to ozone management can readily be identified by their route of transmission and the diseases they can cause. In conjunction, the composition of these viruses have been outlined so that predictions can be made about their relative sensitivity to oxidation by ozone.

### 3.1.1 Enteroviruses

These are small naked viruses that multiply in the gut mucosa and are transmitted from person to person by the fecal-oral route (ingestion disease). The mode of transmission of enteroviruses suggests that these may be suitable target for ozone's virucidal effects. Thus sanitization of aircrafts, hotel facilities, restaurants, public-use areas, daycare centres, nursing homes and pediatric rooms in hospitals may merit serious consideration for enteric viruses. Enteroviruses may be found in the gut of healthy as well as sick children. Common enteroviruses include the following: Polio 1, 2, 3; Coxsackie A 1-24; Coxsackie B1-6; ECHO 1-34; Entero 68-71; Entero 72 (Hepatitis A)

POLIOVIRUS 1, 2, 3—Poliovirus has been well studied and is a good example of an enterovirus, with a RNA-based genome. It is relatively resistant to extremes of pH and temperature, and to lipid solvents and detergents. The only known source is infected man.

After ingestion of the virus, there is local multiplication in the oropharynx, gut mucosa and associated lymph nodes, followed by viraemia (virus circulation in the blood). Occasionally (between 1/100 and 1/1000 of cases) the viraemia may lead to CNS involvement and paralysis.

Virus can be isolated from the throat or stools for some weeks following the incubation period. Immunization can protect individuals from specific strains of poliovirus, but subsequent infection with other strains may still occur. Prior to the introduction of a vaccine (circa 1960) polio was endemic (restricted within a community or population) in the tropics, with rapid circulation in young children (poor hygiene facilitates faecal-oral spread). Universal vaccination commencing in the early 1960s has eliminated polio from the Western world, including North, Central and South America. The disease has also largely been controlled it in Africa and Asia. In South Africa, polio has been effectively eliminated but reintroductions into regions with deficient vaccination programs has resulted in localized outbreaks (e.g. 1982 and 1988). The World Health Organization is forging ahead with a total elimination plan (cf. smallpox) “by the year 2000”. Polio is controlled by: Education, Vaccination and Surveillance.

OTHER ENTEROVIRUSES (Coxsackie, Echo, and Entero 68-72)—Virus structure, epidemiology, pathogenesis of all the enteroviruses are remarkably similar and follow the pattern described for polio. Most infections are silent. Viraemia may lead to degrees of involvement of secondary ‘target organs’ and clinical symptoms and signs related to those organs. For example, the most common type of meningitis seen in some places in the world (such as South Africa) is aseptic meningitis caused by coxsackie or echo viruses (which can often easily be isolated from the CSF, in contrast to polio). Viral meningitis resolves spontaneously without treatment.

HEPATITIS A—It is a small, naked RNA virus particle. Asymptomatic infections are very common, especially in children. Adults, especially pregnant women, may develop more severe disease. Hepatitis describes infections caused by agents whose primary tissue tropism is the liver and Jaundice is the hallmark of infection, but tends to develop late.

It is an enteric virus and enters via the gut; replicates in the alimentary tract and spreads to infect the liver, where it multiplies in hepatocytes. Viraemia is transient. Large quantities of virus are excreted in the stools for two weeks preceding the onset of symptoms. The virus has a worldwide distribution and is endemic in most countries (occurs within pockets of populations).

The incidence in first world countries is declining, with notable exceptions associated with immigration. There is an especially high incidence in developing countries and rural areas.

Transmission includes the following: case-to-case, via faecal-oral route; contamination of food or water with sewage; infected food handlers and shellfish grown in sewage-polluted water.

Prevention includes: passive immunization (normal immunoglobulin given to travelers to third world countries and household contacts of acute cases); and active immunization (inactivated cell culture-derived vaccine has recently become available; not in general use but recommended for travel to certain countries).

HEPATITIS E—This is a Calicivirus; it is a naked virus and contains a RNA genome. The disease is of enteric nature, has a long incubation period 3040 days, and it is acute, self-limiting. It occurs predominantly in young adults between the ages of 1540. Its pathogenesis is similar to hepatitis A; the virus replicates in the gut initially, before invading the liver, and virus is shed in the stool prior to the onset of symptoms. Viraemia is transient. A large inoculum of the virus is needed to establish infection.

TABLE 3.1
Clinical Syndromes Associated with Enteroviruses

| | | Coxsackie | | |
|---|---|---|---|---|
| | Poliovirus | virus | ECHO virus | Enterovirus |
| PARALYSIS-permanent | ✓-1, 2 & 3 | ✓-A7 | | |
| PARALYSIS-temporary | | ✓-B1 to 6 | | |
| MENINGITIS | ✓ | ✓-A and B | ✓ | ✓-71 |
| ENCEPHALITIS | ✓ | | ✓ | ✓-71 |
| RASH | | | | |
| 1) Macular | ✓ | ✓ | ✓ | ✓ |
| 2) Vesicular-(e.g. ‘Hand Foot & | | ✓ | | |
| Mouth Disease’) | | | | |
| SUMMER FEBRILE ILLNESS | ✓ | ✓ | ✓ | ✓ |
| VESICULAR PHARYNGITIS | | ✓-A | | |
| (‘Herpangina’) | | | | |
| MYOCARDITIS | | ✓-B | | |
| EPIDEMIC MYALGIA | | ✓-B | | |
| (‘Bornholm’) | | | | |
| UPPER RESPIRATORY | | ✓-A | ✓ | |
| INFECTION (common cold) | | | | |
| PANCREATITIS | | *✓-B | | |
| GASTRO-ENTERITIS | ✓ | ✓ | ✓ | ✓ |
| CONJUNCTIVITIS | | | | ✓-70 |
| (Haemorrhagic) | | | | |
| HEPATITIS | | | | ✓-72 |
| | | | | (Hepatitis A |
| | | | | virus) |

*Implicated in childhood (insulin-dependent) diabetes.

### 3.1.2 Gastroenteritis

Paediatric diarrhoea remains one of the major causes of death in young children. This is especially the case in Asia, Africa and Latin America where it causes millions of deaths in children aged 0-4. The main factors for high incidence and mortality are unsafe water or inadequate sanitation, requiring social, economic and political solutions. The immediate causes are often of an infectious nature and include a variety of pathogenic micro-organisms including viruses, bacteria and parasites.

A number of different viruses cause diarrhoea, of which the most important is the family of Rotaviruses. Rotaviruses have been estimated to cause 30-50% of all cases of severe diarrhoeal disease in man. In addition to Rotaviruses, two strains of adenovirus (40 and 41) have also been associated with diarrhoeal disease. A group of “small round viruses” (discovered by electron microscopy) have been linked by genetic techniques as closely related to the previously described “Norwalk” agent, are associated with vomiting and diarrhoea.

Apart from the severe problem of diarrhoea in young children, there have been outbreaks of infectious gastroenteritis in adults. Two main groups of virus particles known to be involved sometimes are: (1) Calici viruses (ssRNA) including Norwalk and related agents (‘Hawai’; Ditchling; ‘W’), and (2) “small round viruses” about which very little is known. These do not grow in tissue culture, and are viewed as source of infection through their presence in the electron microscope images of the stool samples.

The mode of transmission of gastroenteroviruses suggests that these may be suitable target for ozone's virucidal effects and thus the sanitizing of aircrafts, hotel facilities, restaurants, public-use areas, daycare centres, nursing homes and pediatric rooms in hospitals.

ROTAVIRUS—The main human pathogens are of Group A subtypes 1, 2, 3, and 4. They are naked RNA viruses. The virus is hardy and may even survive in sewage, despite stringent treatment. The virus is transmitted by faecal-oral route. The incubation period is short (1 to 3 days) and the illness is characterized by sudden onset watery diarrhoea, with or without vomiting that may last up to 6 days (or longer if immunocompromised). The disease is self limiting, but dehydration may result, and this can be severe and life threatening in young children. Modes of prevention include non-specific factors such as improved hygiene, education, and clean water. Breast-feeding helps to provide passive immunity in the newborn (from maternal antibodies). Vaccination is still experimental.

Rotavirus infection is found world-wide and all ages can be infected and reinfection can occur (usually asymptomatic). Maternity hospitals in some countries commonly have resident strains which readily cause asymptomatic infections of newborns. In temperate ‘first world’ populations rotavirus is the main cause of winter gastroenteritis. In tropical and developing countries, rotavirus diarrhoea occurs year round, but peaks in the summer months. However, it is only one of a variety of pathogens causing diarrhoea. In view of the major role of dehydration from diarrhoea as a cause of childhood death, the World Health Organization has waged an intensive campaign for (1) oral rehydration solutions to prevent or treat dehydration and (2) development of a vaccine for rotavirus infections.

ADENOVIRUS—A limited number of strains of adenovirus have been causally related to childhood diarrhoea. Viruses can be isolated from stools, as well as throat and respiratory secretions. The exact role or significance of these strains in the global picture of childhood diarrhoea, especially in developing countries, is not yet fully established.

NORWALK AGENT—Produces ‘Common source’ type of explosive outbreaks of gastroenteritis, with limited secondary spread to household contacts. These often occur in institutions, or follow common source ingestion episodes e.g. celebratory feasts. Vomiting with cramps are more common symptoms than the diarrhoea.

### 3.1.3 Influenza

Influenza viruses are commonly responsible for the flu, and its infection is airborne in nature. These viruses are enveloped and contain RNA as genome. Eight segments of RNA are present and this increases the chance of exchange of segments between strains resulting in the occurrence of new strains making these viruses very difficult to manage. For example, Avian and human strains recombining in pigs in the Far East may permit virulent human strains to evolve. Influenza A virus is essentially an avian virus that has “recently” crossed into mammals. Birds have the greatest number and range of influenza strains. Every 10-15 years a major new pandemic strain appears in man, with totally new proteins that the virus uses to get into cells (antigenic shift). This variant causes a major epidemic around the world (a pandemic). Over subsequent years, this new strain will undergo minor changes (antigenic drift), probably driven by selective antibody pressure in the populations of humans infected. This constant antigenic change means that new vaccines have to be made on a regular basis. The influenza virus case is not unique, but rather it is indicative of viruses' propensity to change and mutate rapidly to adapt to host environment.

New influenza strains spread rapidly in children in schools (and possibly daycares) in places where people crowd together. Influenza epidemics may cause economically significant absenteeism.

Influenza infection is characterized by fever, myalgia, headache and pharyngitis. In addition there may be cough and in severe cases, prostration. There is usually no coryza (runny nose), which characterizes common cold infections. Infection may be very mild, even asymptomatic, moderate or very severe. It is estimated that influenza has resulted in more deaths than deaths from both the world wars combined.

The reservoir is acute infection in other human beings and it is rapidly spread from the reservoir via droplets and fomites with inhalation into the pharynx or lower respiratory tract. The incubation period is short (1-3 days) resulting in rapid spread leading to epidemics. Overall death rates in populations increase in times of influenza epidemics.

Vaccines at best give about 70% protection. They may sometimes not be effective against the most recently evolved strains because the rate of evolution outpaces the rate at which new vaccines can be manufactured. Because another devastating pandemic strain (such as the 1918 pandemic) may appear at any time, the World Health Organization (WHO) maintains worldwide surveillance of flu strains and makes predictions of suitable strains for vaccine production.

Complications tend to occur in the young, elderly, and persons with chronic cardiopulmonary diseases and consist of Pneumonia caused by influenza itself or by secondary infection with bacteria (Haemophilus influenzae, Staphylococcus aureus, Streptococcus pneuminiae) or by other viral superinfection, (eg. Adenovirus).

### 3.1.4 Viral Respiratory Infections

Respiratory infections are grouped into upper respiratory tract infections (URT) that are usually common and fairly mild and lower respiratory tract infections, which can have more severe consequences. In infants and children, URT infections may spread downwards and cause more severe infections and in rare cases even death.

Viral Respiratory Pathogens include: adenoviruses, parainfluenza virus, respiratory syncytial virus, rhinovirus and coronaviruses.

ADENOVIRUSES—These viruses are non-enveloped and contain DNA They cause several syndromes and are spread by droplet, fomites and ingestion. They infect the mucous membranes of the eye, respiratory and gastro intestinal tract, occasionally urinary tract. Local lymph nodes are often involved (enlarged and tender). Infections are usually self-limiting. Adenoviruses may be present in healthy persons, e.g. in stools of children, and may also cause persistent silent infection of the tonsils. There is a wide range of respiratory syndromes associated with Adenovirus. These include infections that are asymptomatic to those that cause pharyngitis, pneumaonia, and acute respiratory syndrome (ARD). The virus is also known to cause epidemic kerato-conjunctivitis (shipyard eye), which is contagious and often spread by multi-shared towels.

ARD is an epidemic form of acute pneumonic disease characteristically appearing in military camps. It has been prevented by enteric capsulation of a live vaccine strain, which bypasses the respiratory tract and sets up a silent infection in the gut, giving protection against acute respiratory infection.

In young children many adenoviruses may cause a generalized infection—upper and lower respiratory tract infection with fever and diarrhoea. Quite separately, some adenoviruses (40141) have been specifically associated with causing acute gastroenteritis in children, which may lead to dehydration and death.

In transplant patients, AIDS or other immunocompromised patients, adenoviruses may cause a variety of infections—renal, disseminated, or a haemorrhagic cystitis.

PARAINFLUENZA VIRUS Types 1, 2, 3 and 4—These can cause minor infections in children and adults. Types 1, 2 and 3 may be associated with more severe lower respiratory tract disease in children. For instance, in an American series of cases, 30% of acute laryngo-tracheo-bronchitis (LTB) cases yielded para-influenza viruses. Type I is especially associated with LTB, sometimes also type 2. Parainfluenza viruses may also cause pneumonia. Under an electron microscope, they are look fairly similar to influenza virus. However, unlike influenza viruses, parainfluenza viruses do not have segmented genomes.

The virus grows locally in the respiratory tract lining of the URT and it may then spread down into the lungs. No specific treatment is available. Killed virus vaccines have been tried but are of limited value. Primary infections usually occur in (early) childhood, with some resultant degree of protection against developing clinical disease later on in life. However, re-infections do occur in adulthood, but disease is subclinical or very minor.

The human parainfluenza viruses are essentially diseases of man only, and are spread by droplets from the nose and mouth to fairly close contacts. Many of them are fairly highly infectious and go around the community in epidemics—often seasonal (winter coughs and colds). Formites might also assist spread.

RESPIRATORY SYNCYTIAL VIRUS (RSV)—This virus is associated with severe pulmonary infections in infants, especially Bronchiolitis. Its composition and mode of transmission is the same as that of parainfluenza viruses.

In Britain, RSV is the single major pathogen in respiratory infections of childhood. The figures from a study in Newcastle are startling. In neonates under 1 year of age, RSV was responsible for:

### 78% of Bronchiolitis

### 38% of Laryngo-Tracheo-Bronchitis

### 36% of Pneumonia

### 35% of Bronchitis

12% of minor respiratory illness

RSV causes a fairly localized infection of the respiratory tract, and infants have no maternal passive protection. An attempted vaccine for RSV was unsuccessful.

RHINOVIRUSES—These viruses are responsible for 50% of common colds. There are over 100 types of rhinoviruses, making it impossible to generate vaccines. They are similar to polioviruses in structure containing genomic RNA and are non-enveloped.

Infection occurs by inhalation of viral particles. Infection is restricted to the upper respiratory tract. The incubation period is short (1 to 3 days) and it is followed by headache, sore throat, fullness in the nose. This is followed by a profuse watery discharge from the nose which gradually thickens and becomes mucopurulent and decreases in volume. The infection resolves in about a week. Following a rhinovirus cold, there is a short period of immunity to all colds. An infected person is infectious in the first two days of coryza (runny nose). Colds are readily acquired from breathing room air from a room crowded with people who have colds.

Complications are usually superinfections by bacteria. A cold may temporarily upset the mucosal cilia and predisposes to secondary invaders especially bacterial infections, e.g. sinusitis (pneumococcus, haemophilus, etc) and bronchitis and possibly pneumonia. These may require antibiotic treatment.

CORONAVIRUSES—These cause 40% of common colds. In animal models, coronaviruses are able to establish persistent infections in the central nervous system. Infection of oligodendrocytes (that make up the sheets that insulate neurons (myelin) and assist in transmission of nerve impulses) leads to demyelinating diseases that have characteristics of human multiple sclerosis.

Coronaviruses contain a single stranded RNA genome and are enveloped. Prior to the emergence of SARS, human coronaviruses received minimal public attention. SARS virus is the most recent human emerging viral disease and its characteristics are described below under “Emerging Viruses of Clinical Importance”.

### 3.1.5 Other Airborne Viruses

VARICELLA-ZOSTER VIRUS—It is one of the seven herpesviruses and the causative agent of chickenpox, which may recur as shingles. This is a common childhood infection that presents as a mild febrile illness associated with a generalized vesicular rash. The incubation period is long, roughly 21 days. Unlike other human herpesviruses, the infection is transmitted either by respiratory droplets or by direct contact with skin lesions. Therefore, sanitization of daycare centres and paediatric rooms in hospitals may merit consideration.

MEASLES, MUMPS AND RUBELLA VIRUS—These are RNA, enveloped viruses. Their mode of transmission is airborne. These viruses are few of the most infectious diseases, and are usually acquired in childhood. Measles and rubella (german measles) infections are characterized by in a red rash. A regiment of vaccine programs for these viruses during infancy and childhood in most developed countries, including Canada, has helped eliminate the risk of infection. However, immigrants and visitors potentially stand a risk of infection.

Measles is also spread by fomites and by respiratory secretions. The virus enters via the respiratory tract or the eye and multiplies in regional epithelial cells. This is followed by viremia and infection of the lymph tissue. Occasionally, measles may result in further complications such as brochopnemonia and encephalomylelitis.

Mumps typically has an acute onset of parotitis. The viruses are transmitted in saliva and respiratory secretions and its portal on entry is the respiratory tract. Viremia follows several days after development of mumps.

Rubella or german measles is spread via respiratory secretions. Rubella infection during pregnancy is known to have devastating effects on the fetus.

### 3.2 Emerging Viruses of Clinical Importance

“Emerging” infectious diseases can be defined as infections that have newly appeared in a population or have existed but are rapidly increasing in incidence or geographic range. Fifty years ago, at the beginning of the anti-microbial and vaccine era, great optimism abounded that the problem of infectious diseases was solved and that the attention of biomedicine should shift to the study of other disease processes. A period of complacency and reduced capacity was followed by the emergence of new catastrophic infectious diseases like influenza, AIDS and SARS. These catastrophes have raised international awareness of the importance of establishing appropriate surveillance mechanisms and to be prepared to response quickly to these diseases. The fact that developing vaccines or appropriate antiviral therapies for these diseases is a long (and often unfruitful) endeavor, underscores the importance of developing innovative ways of inactivating viruses (for example, ozone-induced viral inactivation) before additional cycles of infection occur from exposure to contaminated areas or facilities.

### Factors Contributing to Emerging Viral Infections

Specific factors precipitating disease emergence can be identified in virtually all cases of communicable (infectious) diseases. These factors (Table 3.2) are increasing in prevalence which, together with the ongoing evolution of viral and microbial variants and selection for drug resistance, suggests that infections will continue to emerge and probably increase. These are global problems, as demonstrated by influenza, HIV/AIDS, West Nile disease and most recently SARS. Under suitable circumstances, a new infection first appearing anywhere in the world could traverse entire continents within days or weeks.

TABLE 3.2
Recent examples of emerging viral infections and probable factors in their
emergence

| Infection or Agent | Factor(s) contributing to emergence |
|---|---|
| Argentine, Bolivian hemorrhagic fever | Changes in agriculture favoring rodent host |
| Bovine spongiform encephalopathy (cattle) | Changes in rendering processes |
| “Mad Cow Disease” - Prions* | |
| Dengue, dengue hemorrhagic fever | Transportation, travel, immigration and migration; |
| | urbanization |
| Ebola, Marburg | Unknown (in Europe and the United States, |
| | importation of monkeys) |
| Hantaviruses | Ecological or environmental changes increasing |
| | contact with rodent hosts |
| Hepatitis B, C | Transfusions, organ transplants, contaminated |
| | hypodermic apparatus, sexual transmission, |
| | vertical spread from infected mother to child |
| HIV | Migration to cities and travel; after introduction, |
| | sexual transmission, vertical spread from infected |
| | mother to child, contaminated hypodermic |
| | apparatus (including during intravenous drug use), |
| | transfusions, organ transplants |
| HTLV | Contaminated hypodermic apparatus |

TABLE 3.2
Recent examples of emerging viral infections and probable factors in their
emergence

| Infection or Agent | Factor(s) contributing to emergence |
|---|---|
| Influenza | Possibly plg-duck agriculture, facilitating |
| (pandemic) | reassortment of avian and mammalian influenza |
| | viruses |
| Lassa fever | Urbanization favoring rodent host, increasing |
| | exposure (usually in homes) |
| Rift Valley fever | Dam building, agriculture, irrigation, possibly |
| | change in virulence or pathogenicity of virus |
| Severe Acute | Infected exotic wild animals kept for food |
| Respiratory | (Himalayan palm civets and a raccoon dog). |
| Syndrome | |
| (SARS) | |
| West Nile Virus | Global warming due to deforestation and pollutants |
| Yellow fever | Conditions favoring mosquito vector (in new areas) |

*Prions are not actually viruses, but rather protein containing infectious agents which do not have a nucleic acid (DNA or RNA);. the protein itself is the infectious agent. Prions infect hosts and use the host cell machinery to facilitate replication just like viruses. Bovine spongiform encephalopathy or the mad cow disease is a prime example of a prion.

The emergence of new viruses is a trend that is likely to continue. One factor driving the emergence of new viruses is ecological change, including habitat encroachment, climate change, and the widespread use of vaccines and other antiviral agents leading to the evolution of new, resistant viruses. Also a factor is human demographics, including increased population, and increased migration and immigration. As new human populations develop in previously uninhabited areas, and as diverse populations come in contact with each other through immigration, the potential for viral spread increases. Another factor is increased international travel, which can lead to the rapid spread of disease worldwide, as was illustrated by the SARS epidemic. Finally, increased global food trade has the potential to lead to the spread of new and emerging viruses through the human food chain.

Identification of these factors is critical for the future management of emerging infectious diseases. Most of these activities cannot be reversed, therefore, there is a need for better surveillance mechanisms and equally importantly, is the need for development of innovative techniques to eliminate the emergent pathogen prior to development of vaccines and antivirals (which currently tend to be lengthy processes).

### 3.3 Virucidial Activity of Ozone—Historical Perspective

There have been many studies that show anecdotal of ozone as an antiviral agent. The table below summarizes a number of them:

TABLE 3.3
A summary of studies that have tested ozone as an antiviral agent.

| | | Duration | Concentration | | |
|---|---|---|---|---|---|
| Species | Method of | of Ozone | of | | |
| of Virus | Application | Exposure | Exposure | Effectiveness | References |
| Polio-virus | Ozone was | 20 seconds | ozone | >99.99% of MS2 coliphage | Finch GR, |
| type 3 & | added as a side | | demand- | inactivated, 1.6 log units more | Fairbairn N. |
| MS2 | stream from a | | free 0.05 M | inactivation was observed | |
| coliphage | concentrated | | phosphate | with MS2 coliphage than with | |
| | stock solution | | buffer (pH | poliovirus type 3 | |
| | (aqueous) | | 6.9) at 22 | | |
| | | | degrees C. | | |
| Polio- | Ozone used to | | 1.0 to 1.5 mg | Dependant on the condition | Thraenhart O, |
| viruses | inactivate viruses | | ozone/liter | of the water (redox-potential, | Kuwert E. |
| | in water supply | | (dissolved) | pH etc.) | |
| | (aqueous) | | | | |
| Polio-virus | Ozone steadily | | 0.25 to | | Herbold K, |
| 1 & | flowing water at | | 0.38 mg/l | | Flehmig B, |
| hepatitis A | 20 degrees C. and | | for | | Botzenhart K. |
| virus | pH 7 (aqueous) | | complete | | |
| | | | inactivation | | |
| | | | of HAV; | | |
| | | | 0.13 mg/l | | |
| | | | for | | |
| | | | complete | | |
| | | | inactivation | | |
| | | | of PV1 | | |
| Hepatitis A | Single-particle | 60 seconds | 1 mg/L or | complete (5 log) inactivation | Vaughn JM, |
| virus | virus preparations | | greater at | | et al. |
| | suspended in | | all pH | | |
| | phosphate- | | levels | | |
| | carbonate buffer | | | | |
| | (aqueous) | | | | |
| Enteroviruses | Enteroviruses in | | | inactivation rate of | Ivanova OE., |
| | sewage exposed | | | enteroviruses directly | et al. |
| | to ozone | | | depended upon the dose of | |
| | | | | ozone and time of contact | |
| | | | | with it | |
| Human & | Single-particle | | 0.25 mg/ | both virus types were rapidly | Vaughn JM., |
| simian | virus stocks | | liter or | inactivated | et al. |
| rotaviruses | exposed to | | greater | | |
| | dissolved ozone | | | | |
| | (aqueous) | | | | |
| HIV Type 1 | Ozone continually | 2 hours | 1,200 ppm | >11 log inactivation (NB. | Wells KH., |
| | delivered to fluids | | | Minimal effect on factor VIII | et al. |
| | containing HIV | | | activity in both plasma and | |
| | Type 1 | | | immunoaffinity-purified | |
| | | | | preparations) | |
| Human | Viruses isolated | | | Human rotavirus was at least | Harakeh M, |
| rotavirus, | from faeces and | | | as resistant as poliovirus, | Butler M. |
| SA11 & | resuspended in | | | coxsackievirus, echovirus and | |
| other | wastewater | | | f2 coliphage and was | |
| enteric | effluent were | | | strikingly less sensitive to | |
| viruses | exposed to ozone | | | inactivation than the simian | |
| | (gaseous) | | | rotavirus | |
| Venezuelan | Ozone in liquid | 45 minutes | 0.025 mg | Inactivation of 10 (6.5) median | Akey DH, |
| equine | phase application | | of ozone | cell culture infective doses | Walton TE. |
| encephalo | was applied to | | per liter | from control levels of 10 (7.25-7.5) | |
| myelitis | viruses (aqueous) | | | represented a reduction | |
| virus | | | | of 99.99997% of the viral | |
| | | | | particles | |
| Poliovirus | Ozone demand- | Step 1: | 0.1-2.0 mg/l | Step 1: 95 to 99% of the virus | Katzenelson E., |
| | free water with a | 0.2-1.0 s; | | was inactivated | et al |
| | fast-flow mixing | Step 2: | | Step 2: remainder inactivated | |
| | apparatus | several | | | |
| | (aqueous) | minutes | | | |
| HIV | Serum and | | 0.5 to 4.0 micrograms/ml−1 | Complete inactivation of HIV | Carpendale MT, |
| | serum- | | | obtained at 4.0 micrograms/ml−1 | Freeberg JK. |
| | supplemented | | | | |
| | media were | | | | |
| | treated with | | | | |
| | ozone (aqueous) | | | | |
| HEp-2 | Exposure of viral | 30 seconds | 4.06-4.68 mg/ | Inactivated cell-associated | Emerson MA, |
| cell- | samples to a | | liter | poliovirus and coxsackievirus | Sproul OJ, |
| associated | continuous-flow | | | | Buck CE. |
| poliovirus | ozonation system | | | | |
| (Sabin 1) & | (aqueous) | | | | |
| coxsackievirus | | | | | |
| A9 | | | | | |
| Influenza | Patients suffering | | 0.5 ppm | Severity of infection reduced | Jakab GJ, |
| virus | from influenza | | | | Hmieleski RR. |
| | exposed to ozone | | | | |
| | (gaseous, in vivo) | | | | |
| Norwalk | Viruses in water | Up to 5 min | 0.37 mg/l | Reductions of Norwalk virus | Shin GA, |
| Virus, | exposed to | | | were >3 log(10) within a | Sobsey MD. |
| Poliovirus | dissolved ozone | | | contact time of 10 s, and | |
| 1, and | (aqueous) | | | these were similar to the | |
| Bacterio- | | | | reductions of the other two | |
| phage | | | | viruses determined by the | |
| MS2 | | | | same assay methods | |
| Human | Viral suspensions | | 25 μg/ml | reased rotavirus infectivity by | Khadre MA, |
| rotavirus | exposed to ozone | | | 8 to 9 log10 TCID50/ml. | Yousef AE. |
| | (aqueous) | | | | |
| HVJ*, | Viral suspensions | 1 hour | 100 ppm | TMEV reduced to 0; HJV and | Sato H, |
| TMEV^{†}, | were exposed to | | with high | MHV were even more | Wananabe Y, |
| Reo type 3 | gaseous ozone | | humidity | susceptible than TMEV, | Miyata H. |
| virus (RV) | (gaseous) | | | whereas RV was the most | |
| & MHV^{‡} | | | | resistant strain. | |
| Vesicular | Virus-spiked, | | 3 to 14 mL | Inactivated by greater than 4 | Wagner SJ., |
| stomatitis | dilute, red cell | | of 1.4 mmol/L | log10 | et al. |
| virus | suspensions were | | (65 micrograms/mL) | | |
| (VSV), | exposed to ozone | | to | | |
| bacterio- | (aqueous) | | 1.6 mmoler/L | | |
| phage phi 6 | | | (75 mg/mL) | | |
| T1 phage | Virucidal | 10 minutes | 10 ppm | 90% reduction in number of | Murakami H., |
| | effectiveness of | | | phages | et al. |
| | denture cleaner | | | | |
| | that uses ozone | | | | |
| | (gaseous) | | | | |
| Rhinovirus | Volunteers | 6 hours per | 0.3 ppm | No difference in viral titres | Henderson FW, |
| | experimentally | day for five | | between experimental and | et al. |
| | inoculated with | days | | placebo groups; no adverse | |
| | type 39 rhinovirus | | | effects in experimental group | |
| | were exposed to | | | | |
| | inhaled ozone | | | | |
| | (gaseous, in vivo) | | | | |
| Influenza | Mice infected with | | 0.5 ppm | Exposure to ozone resulted in | Wolcott JA, |
| virus | influenza virus | | | less widespread infection, as | Zee YC, |
| | were exposed to | | | well as altered the distribution | Osebold JW. |
| | inhaled ozone | | | of viral antigen (NB. No | |
| | (gaseous, in vivo) | | | difference in viral titres, but | |
| | | | | rather in viral distribution, | |
| | | | | which interestingly reduced | |
| | | | | severity of illness) | |
| Respiratory | Human alveolar | 2 hours | 1 ppm | No difference in the | Soukup, et |
| syncytial | macrophages | | | percentage of cells infected | al., 1992 |
| virus | exposed infected | | | was observed between the | |
| | with syncytial | | | experimental and control | |
| | virus (RSV) were | | | groups, nor was any | |
| | exposed to ozone | | | difference observed in the | |
| | (gaseous, in vitro) | | | amount of infectious RSV | |
| | | | | produced | |
| Influenza | Mice infected with | Continuous | 0.5 ppm | Ozone exposure did not alter | Jakab GJ, |
| virus | influenza were | | | the proliferation of virus in the | Bassett DJ., |
| | continuously | | | lungs as quantitated by | 1990 |
| | exposed to ozone | | | infectious virus titers, but | |
| | (gaseous, in vivo) | | | mitigated the virus-induced | |
| | | | | acute lung injury by | |
| | | | | approximately 50%. Ozone | |
| | | | | exposure mitigates acute | |
| | | | | virus-induced lung injury & | |
| | | | | residual lung damage. | |
| Murine | Infected mice | Continuous | 0.5 ppm | Reduced the severity of the | Jakab GJ, |
| influenza | were exposed to | | | disease | Hmieleski RR., |
| A/PR8/34 | gaseous ozone | | | | 1988 |
| | during the course | | | | |
| | of infection | | | | |
| | (gaseous, in vivo) | | | | |
| Influenza | Mice infected with | 3 hours | 0.6 ppm | Complete inhibition of virus | Fairchild, |
| virus | influenza inhaled | (post | | growth in nose | 1977 |
| | gaseous ozone | infection) | | | |
| | (gaseous, in vivo) | | | | |
| Hepatitis A | Closed-circuit | 24-48 | | From 1.72 log 50% tissue | De Medici, |
| virus | depuration | hours | | culture infective dose | et al., 2001 |
| | system using | | | [TCID50] ml(−1) to <1 log | |
| | both ozone and | | | TCID50 ml(−1) within 24 h; | |
| | UV light | | | and from 3.82 log TCID50 | |
| | | | | ml(−1) to <1 log TCID50 ml(−1) | |
| | | | | within 48 h | |

*HVJ is hemagglutinating virus of Japan
^{‡}MHV is murine hepatitis virus
^{†}TMEV is Theiler's murine encephalomyelitis virus

Clearly, a large number of studies have been conducted on the antiviral properties of ozone, and they can be classified as either using aqueous or gaseous ozone. There does not appear to be any quantifiable relationship between the method of application and the antiviral capacity of ozone. Rather, the virucidial activity of ozone in any form is dependent on a combination of its concentration and the duration of ozone exposure. Not surprisingly, the extent of viral inactivation is directly proportional to these factors.

Having said this, however, it is still possible to infer a few trends from the available data. Although it is not possible to say for certain, it does appear that aqueous ozone is far more effective at achieving viral inactivation than gaseous ozone. The studies involving aqueous ozone all achieved significant viral inactivation in short time periods, ranging from 20 seconds to five minutes (although two studies were conducted for 45 minutes), and the concentration of aqueous ozone ranged from 0.1 mg/l to 4.68 mg/l.

Gaseous ozone, in contrast, was generally used for much longer periods of time. The duration of exposure for gaseous ozone ranged from 1-3 hours, and some studies even used gaseous ozone continuously, or over a period of several days. The concentration of gaseous ozone used ranged from 1 ppm-100 ppm (although one study did use a concentration of 1200 ppm). The following table summarizes the differences between gaseous and aqueous ozone:

TABLE 3.4
The range of the length of ozone application and concentration of ozone
necessary to attain significant viral inactivation.

| Method | | |
|---|---|---|
| of Application | Duration of Application | Concentration of Ozone |
| Aqueous | 20 seconds-five minutes | 0.1 mg/l 4.68 mg/l |
| Gaseous | 1-3 hours | 1 ppm-100 ppm |

Some of the above studies, for example for polio and hepatitis A, have been conducted in aqueous applications. Though these show positive results, they do not address susceptibility of the viruses to application of gaseous ozone. Standardized experiments are required to directly compare susceptibility of each virus type.

As measured against the public health standards for indoor ozone levels discussed in section 2.1.4, the concentrations of gaseous ozone used in the studies discussed above far exceeded recognized health standards, by a factor of 5-10 times. In other words, if used at concentrations that do not exceed public health standards, ozone applied to indoor air may not effectively remove pathogens. There are very few studies examining the effect of gaseous ozone at lower concentrations for shorter time periods and it is recommended that ozone's effectiveness be tested in these lower ranges (however, one study did find that 0.3 ppm ozone had no effect on individuals infected with rhinovirus). A caveat should be included that the study involving rhinovirus was done in vivo, and therefore does not necessarily indicate that ozone at such a low concentration would be ineffective against rhinovirus. Further in vitro studies are required to confirm the concentration at which ozone will have an inhibitory effect on rhinovirus in the environment.

If it is found that gaseous ozone is ineffective at lower levels, then it may be necessary to develop an alternative method of ozone application that is effective at far lower concentrations and for far shorter time periods. One such potential method of delivery is through the nebulization of ozone, which is considered below in the Prospective View section

Interestingly, some viruses appear to be more resistant to ozone than others. Based on the studies above, the order of resistance to ozone among viruses, from least resistant to most resistant, is detailed in the following figure:

Admittedly, this figure is based on only a few references, and therefore may not be entirely accurate, but it does appear that human rotavirus is among the most resistant viruses, while viruses such as bacteriophage are among the least resistant. Therefore, it would seem that any potential application of Treated Air System's products should be geared towards inactivating viruses at the most resistant end of the spectrum.

Generally speaking, anecdotal studies have shown ozone to have virucidal properties, however, its efficacy is dependent on several factors, including the mode of application (gaseous or aqueous), the concentration of ozone, the virus type, and exposure time. Further standardized in vitro studies are required to compare the susceptibility of appropriate viruses such as enteric, gastroenteric, respiratory and airborne in nature, to ozone application.

### 3.4 Virucidial Activity of Ozone—Prospective View

There are a number of novel ways in which ozone is being suggested for use as an antiviral agent, ranging from decontamination of foodstuffs to the inactivation of novel pathogens such as SARS virus and even prions such as BSE. The table below summarizes several possible novel applications of ozone:

TABLE 3.5
Some potential applications of ozone as an antiviral agent.

| Novel Use of | | |
|---|---|---|
| Ozone | Method of Application | References |
| Against SARS | Could theoretically be applied to medical equipment and hospital | Sunnen, 2003, |
| virus | rooms in gaseous form, or inhaled by patients infected with SARS | Lemmo, 2003 |
| | virus | |
| Decontamination | Food exposed to ozone in gaseous or aqueous phases; Use of | Kim et al., 2003; |
| of foodstuffs | ozone to sanitize equipment, packaging materials, and | Majchrowicz, |
| | processing environment is currently investigated; The food | 1998 |
| | industry also is interested in using ozone to decontaminate | |
| | processing water and decrease its chemical and biological | |
| | oxygen demand. This application improves the reusability of | |
| | processing water and allows for environment-friendly processing | |
| | operations | |
| Inactivation of | Recent testing on an animal prion model using a sterilization | Agri-Food |
| infections | process developed by the Canadian company, Technologies of | Surveillance |
| prions* | Sterilization With Ozone (TSO3), Inc. has shown promising | Systems Branch: |
| | results for inactivating infectious prions. Ozone has the potential | Newsletters, Vol. |
| | to completely eliminate infectious prions due to its intense | 1 No. 11 Jun. |
| | oxidizing action, which is able to break chemical bonds. | 2002 |
| | Therefore, it can permanently alter the protein structure of the | |
| | prion, rendering it inactive and unable to infect. Its unique | |
| | capabilities allow ozone to destroy these small, infective prions, | |
| | while leaving the much larger protein and lipid molecules found in | |
| | the blood of mammals, including cattle and humans, functionally | |
| | intact. | |
| Nebulization | Nebulization technique (could be used to inactivate viruses in | Kekez MM, Sattar |
| technique of | large volumes of body fluids, such as plasma, partial blood and | SA. |
| ozone | perhaps whole blood). For the method of nebulization, the | |
| application | exposure time of droplets with ozone is a few seconds, whereas | |
| | for the thin film method the exposure time is measured in hours. | |
| Application of | Gaseous ozone applied to blood, serum, and other biological | Medizone |
| gaseous ozone | fluids in precise concentrations as measured by the equipment | International |
| in precise | sold with the ozone generator. The delivery of ozone in precise | http://www.medizoneint.com/scienceframe.html |
| concentrations | concentrations allows ozone to be used in optimal concentrations | |
| | within a, “therapeutic window” in which viruses may be inactivated. | |
| | but other, essential biological components such as hemoglobin | |
| | are not inactivated. | |
| The sterilization | TSO3, a Quebec-based company, uses ozone as a sterilizing | TS03 Press |
| of medical | agent, and sells hospital sterilization units. The company claims | Releases |
| equipment | that the ozone sterilization process is a safe, efficient, fast and | www.tso3.com |
| | cost-effective response to evolving sterilization needs. One of | |
| | their products, the 125L Ozone Sterilizer, was licensed by Health | |
| | Canada in May 2002, and they are currently awaiting Food and | |
| | Drug Administration (FDA) approval, for which the company filed | |
| | a 510(k) application in March 2002. In November 2002, the | |
| | company signed an agreement with Vancouver General Hospital | |
| | to pilot test its equipment. | |

*Prions are not actually viruses, but rather protein containing infectious agents which do not have a nucleic acid (DNA or RNA);. the protein itself is the infectious agent. Prions infect hosts and use the host cell machinery to facilitate replication just like viruses. Bovine spongiform encephalopathy or the mad cow disease is a prime example of a prion.

While it is important to note that the effectiveness of the above applications is purely speculative, many of these applications do merit consideration. The sterilization of medical equipment in particular is an excellent application for ozone, as ozone can be used at concentrations as high as necessary and leaves a non-toxic residue at completion. Additionally, the decontamination of foodstuffs may be a potential application of ozone, for much the same reasons as the sterilization of medical equipment.

The nebulization technique discussed above is unique for its ability to significantly reduce the duration of ozone exposure necessary to obtain substantial viral inactivation, over another “film” method. However, only the abstract for this paper could be obtained, and so it is difficult to speculate as to whether the nebulization of ozone could indeed compete with gaseous application.

### 4.0 PILOT MARKET ANALYSIS

Ozone has been long known for its ability to neutralize toxic gases, decontaminate air and water, and disinfect pathogens. These unique properties have led to multiple exploitation of ozone in therapeutics; sanitation of public-use areas such as toilets, decontamination of water, decontamination of indoor air in public-use areas, nursing homes and operating rooms, sterilization of food and in packaging, fumigation of homes and building (sick building syndrome) and disinfection of large scale air conditioning systems in hospitals (Rice, 2002). TASM recognizes the potential market of ozone as an antiviral agent and is interested in pursuing it in the sanitation markets in the hospitality and aircraft industries, and perhaps as well as several other industries including hospitals. Because ozone is a gas, unlike other disinfectants, it has the advantage of spreading itself easily and entering small spaces. In addition, it has a short half-life and can be considered environmentally friendly. However, given that ozone is a lung irritant and that studies suggest that ozone may react with chemicals normally present in indoor environments to form harmful byproducts, further research is needed to study the feasibility and safe use of ozone as an antiviral agent.

Most viruses are sophisticated entities that continually evolve and develop to adapt to their host environment using biological strategies such as mutation, which leads to genetic diversity. The SARS virus, a coronavirus, has recently received much attention as the newest emerging infectious agent of global importance. Recent evidence suggests that the source of SARS virus may have been the wild cat consumed for food (www.english.peopledaily.com).

The aggressive spread of SARS from Asian countries to other countries including Canada has challenged the airline, hospitality and tourism industries and the hospitals. The spread of SARS has had a devastating effect on the affected country's economy. The challenge of SARS has forced global health organizations and many countries to rethink their strategies on containing the global spread of diseases, especially enteric viruses such as polio, which spread readily through coughing, sneezing, mucous droplets, fecal contamination, etc. and are thus are difficult to contain. The governments of Canada and Hong Kong have spent millions of dollars to stop the spread of SARS and to support their affected economies.

SARS, of course, is not the only virus of concern. A variety of airborne, gastroenteric and enteric viruses, including varicella zooster (chicken pox), measles virus, rhinovirus (cold), influenza virus (flu), poliovrus, rotavirus, hepatits A, norwalk virus and adenovirus, all represent risks in terms of contagiousness and infectivity.

For each potential market considered below, it will be necessary to thoroughly investigate the efficacy and feasibility of the use of ozone air treatment systems. The feasibility investigation should ensure that human exposure to ozone is limited due to its potential side effects on lung and asthma (Ross et al., 2002) but also that the highest possible strength of ozone required to eliminate the most virulent and resistant transmittable viruses is used. Such studies would examine the effects of ozone concentration, time, and treatment frequency to determine the optimal levels of ozone delivery necessary.

Although high concentrations of ozone are sometimes used to help decontaminate unoccupied spaces from certain chemical and odour contaminants, little is known about the chemical by-products left behind by these processes. Ozone can also adversely affect indoor plants, and damage materials such as rubber, electrical wire coatings, and fabrics and art work containing susceptible dyes and pigments (www.epa.gov/iedweb00/pubs/ozoneaen). Feasibility studies should also include research that needed to more completely understand the complex interactions of indoor chemicals and compounds in the presence of ozone, especially in delicate surroundings such as aircraft and their components, as well as hospitality and other public-use areas by humans and animals.

### 4.1 Airline Industry

Recently, particularly in the wake of the SARS outbreak, there has been a growing concern that both passengers and crew-members may be exposed to high risk transmission from other infected passengers during flight on aircraft. The WHO and other national agencies have provided guidelines to reduce this risk. These include stringent screening of potentially infected passengers, sanitation controls in the aircraft, air decontamination, and procedures in a case an infectious passenger is diagnosed with a history of air travel, including tracing and screening of contacts for possible interventions.

Despite the thoroughness of these standards, it is impossible to completely screen for all infected passengers. There are several reasons for this. Many Asian airlines, for instance, which have implemented the screening procedures, failed to prevent infected passengers from boarding, as many infected passengers do not show any symptoms during the incubation period of the virus. And as long as the frequency of global flights continues to increase, and as long as the access to more exotic destinations gets easier, previous boundaries will disappear allowing hidden and new diseases to emerge and spread more readily.

Although ozone disinfection can not be utilized to eliminate the risk of viral transmission within the aircraft during flight, decontamination of the entire aircraft chamber by fumigation immediately with ozone after the unloading of passengers could potentially provide preventative measures and help safeguard the janitors, staff and the next batch of passengers and crew-members boarding the aircraft from contracting an infection. The fumigation procedure could also include treatment of the air conditioning systems. The potential of these preventative measures merit further investigation.

In addition to its antiviral properties, ozone has a number of pleasant corollary effects that may also be a boon. Ozone, due to its powerful oxidizing strength, can help to remove many odors.

Additionally, the fact that an airline goes to the added trouble of ozone decontamination can certainly be a positive marketing feature. However, it is important that feasibility studies are done to evaluate the potential of risks to delicate surroundings in the aircraft.

### 4.2 Hospitality Industry

Many of the same individuals who travel via airlines also stay in hotels; there is a large overlap between the two populations. In a hotel setting, it is virtually impossible to screen infected guests and prevent them from staying in a hotel room and spreading infection to other guests and hotel staff. And although individuals in hotels stay in separate rooms, there is still a strong risk of infection being passed between hotel guests. Witness the pattern of transmission of SARS, which was passed from one hotel guest to another staying at Hong Kong's Metropole hotel (FIG. 4.1).

Although ozone decontamination cannot eliminate the risk of viral transmission to staff and other guests during the stay period of the infected guest, it may help to prevent and safeguard to some extent the spread to janitors and to subsequent guests living in the hotel room. The cleaning procedure of a room could commence with fumigation with ozone, followed by a period of exposure to ozone. Periodic fumigation of the central air conditioning system may also serve as an additional precaution. These preventative measures could potentially safeguard the cleaning staff and next guests, and this makes the ozone technology in the hospitality industry worth further investigation. As well, hotel conference rooms and rooms where other venues take place could also be sanitized using ozone, allowing large-scale conventioneers to have more confidence in the cleanliness of their surroundings. Furthermore, as with airlines, ozone has the potential to reduce odors and tourists and customers may find a sanitized room is more appealing and would serve as a good marketing strategy. Finally, it should be noted that this sort of application for ozone need not be limited to hotels. Other guest areas, such as cruise ships and time share properties, could also make use of ozone technology.

It should be noted that there is some precedent for the use of ozone in hotels. During the recent SARS scare, one Bangkok Hotel, the Conrad, took the precaution of installing ozone treatment air-conditioning systems in several public areas, including restaurants. Additionally, conversations with a few British Columbia hotel owners revealed that they currently use ozone to remove odors from rooms, especially in cases where smokers had smoked in non-smoking rooms. According to these owners, the use of ozone in this manner is standard practice in British Columbia and much of North America (personal communication, 2003). However, feasibility studies are required to evaluate the management of potential risks for ozone use in the hospitality industry.

### 4.3 Hospitals, Nursing Homes, Daycares, and Laboratories

Hospitals, Nursing Homes, Daycares, and Laboratories are high risk areas for the transmission of enteric, gastroenteric and airborne viruses, such as polio, Hepatitis A, rotaviruses, SARS virus, varicella zoster (chicken pox), measles virus, rhinovirus (cold), influenza virus (flu), RSV, Norwalk virus and adenovirus. Both patients and staff are at risk of contracting many of these diseases.

Individuals are often forced to spend hours in hospital waiting rooms along with other sick individuals, allowing diseases to multiply and migrate. Although ozone cannot be used to prevent the spread of disease between individuals, it can be used in a number of other ways. It would be prudent to fumigate a hospital room between patients, especially in cases where a hospital acquired infection could possibly occur or in rooms that harboured patients with contagious disease, or in rooms that will be inhabited by immunocompromised patients. It should be noted that Infants and elderly people are also particularly vulnerable to hospital-acquired infections. Preference should be given to paediatric and geriatric wards. Additionally, ozone could also be used to clean the ventilation systems of hospitals, which would help to stem the spread of viruses throughout the hospital.

Gastroenteric and chickenpox infection with rota and varicella zoster viruses, respectively, are common problems at daycares. Fumigation of daycares and the ventilation system after hours could help eliminate lingering viruses.

### 4.4 Sanitization of Industrial Fabric

In addition to the use of ozone as an environmental disinfectant, there is another application for ozone in hotels, hospitals, cruise ships, and even airlines. A number of companies, such as IndustrOzone Technologies, L.C., are marketing “ozone washers” which require less water, no chemical additives, and are more environmentally friendly and often cheaper than conventional washers. Add to this the fact that ozone has strong antiviral properties, and the potential of ozone as a laundering agent has significant potential. Finally, there may also be the potential for certain tax incentives or socially-conscious marketing incentives for the use of ozone laundry systems, due to their environmentally-friendly nature. One such program is in existence in California, and in Canada—which has ratified the Kyoto Protocol—such an initiative may fall within the framework of the protocol, although more investigation would be needed to determine whether this is in fact the case.

### 4.5 Public Washrooms, Physicians' Waiting Rooms, Gyms and Locker Rooms, Airports, Tourist Areas

As with the airlines and hospitality industries, areas such as public washrooms, physicians' waiting rooms, gyms and locker rooms, and airports could benefit from ozone fumigation if not daily, at least regularly. Due to the public nature of all of these places, diverse groups of people, some of whom may be carriers of disease—congregate and the potential for infection is high. While ozone would not be capable of preventing the transmission of infection from person to person, it could be used as a decontaminant to clean surfaces (in either gaseous or aqueous form) and thus preventing the buildup of viral particles.

### 4.6 Medical Equipment and Medicine

There are already a number of manufacturers using ozone to disinfect medical equipment. The sterilization of medical equipment is an especially appropriate application for ozone because it can help to replace some of the less effective, yet more hazardous chemicals currently used in the sterilization of medical equipment.

One company, Medizone International (www.medizoneint.com) has proposed the potential use of ozone as an antiviral agent in the processing of blood and serum products. This technology is currently under investigation for future use.

### 4.7 Granaries, Sawmills, and Scientific Research Involving Rodents (for Hantavirus)

According to the Canadian Centre for Occupational Health and Safety, areas such as granaries and sawmills, where rodents can thrive, and even areas such as scientific laboratories with high numbers of rodents, are areas where hantavirus can accumulate. Workers in these areas are at risk of contracting hantavirus, which is deposited in rodent fecal matter and can become airborne and inhaled. The application of gaseous ozone to these areas could significantly reduce the levels of hantavirus, and create a safer working environment. Additionally, ozone would have the pleasant benefit of eliminating much of the odors that plague areas such as these.

4.8 Other applications

Although ozone would not prevent the organism-to-organism transmission of infectious particles, it would help to inactivate infectious before they could be transmitted.

Food processing & meat packing—There are a number viruses which can be transmitted via foodstuffs, including many enterc viruses. The potential efficacy of ozone in decontaminating foods has been investigated by a number of sources, and this may also represent a potential market for ozone. Theoretically, ozone could be used in either aqueous or gaseous form to disinfect food or surfaces used to prepare foods and in packaging meats. As well, ozone's strong oxidizing power has been shown to strip chemical residues such as pesticides from food.

Barns, Ranches and slaughterhouses—The recent news has shown, the risk of the prion-associated “mad cow disease” (BSE) may also be present in areas such as these. The is a strong potential for the use of ozone as a disinfectant in these areas, as it would have the dual effect of both acting as an antiviral agent and also of greatly reducing the infectivity of prions such as BSE.

Veterinarians and Zoos—Decontamination of cages, animal rooms and contaminated surfaces could potentially help spread of viruses within the animals.

### 5.0. CONCLUSIONS & RECOMMENDATIONS

It is clear from anecdotal studies available that ozone has efficacy as an antiviral agent, however the precise boundaries of its effectiveness are unknown. Further in-vitro studies are required and these should include standardized experiments with control and experimental groups to address: optimal concentration; duration of application; method of application; susceptibility of enteric, gastroenteric, respiratory and airborne viruses; and application in conjunction with other disinfectants.

There is some controversy over the use of ozone as an effective antiviral agent and the health hazards that ozone poses to humans. Ostensibly, the levels of ozone that is required to achieve significant viral inhibition far exceed the highest levels of ozone recommended by human health standards. Furthermore, it is clear that the reactivity of ozone is not limited to biological substances. Many of the non-biological substances that are present in normal indoor environments, including many chemicals present in new carpets, have been shown to react with ozone to produce harmful byproducts. Studies should also be extended to include feasibility of ozone application in specific industry surroundings, especially in aircraft cabins where the surroundings are obviously delicate.

For health reasons, ozone use in occupied spaces is restricted and its gaseous application should be followed by sufficient time to permit for ventilation, dissipation and disintegration. Both the airline and hospitality industries thrive on high occupancy rates and high turnaround times, particularly during peak travel season. Hotels have tight schedules for checking out guests, cleaning the rooms, and checking in new guests. Similarly, airlines are under pressure to deboard passengers, clean and refuel, and take off again with a new complement of passengers. This makes it difficult to apply ozone at levels approaching antiviral effectiveness in hotels and airlines during the short unoccupied times. Ironically, both industries are susceptible to the spread of viral infection. Ozone application has already entered the hospitality industry as an odour neutralizer. The effectiveness and the optimal conditions for viral efficacy in hotel rooms are unknown. In light of the recent SARS crisis where hotels have proven to be potential threats in viral disease spread, the mere hint of the potential antiviral property of ozone could add value to its application in the hospitality market. As for its application in the airline industry, it would be difficult to use gaseous ozone as a disinfectant in aircraft cabins. The time pressure faced by the aircraft industries and the potential risks of oxidizing properties of ozone to delicate surroundings in the aircraft may negate the use of ozone at concentrations that would (based on available data) inhibit viral growth.

Nevertheless, further research for application of ozone as an antiviral is still recommended, for several reasons. First of all, the anecdotal data currently available is far from complete. It may well be possible that ozone could be applied in far lower concentrations, or for far shorter duration, and would still maintain a significant degree of antiviral effectiveness. Secondly, further research may reveal novel methods of application of ozone (for example, co-application of ozone and other chemical agents) that would maintain ozone's level of antiviral effectiveness and mitigate many of ozone's harmful side effects.

There are a number of other potential applications for ozone generators in the hospitality and airline. Ozone may be effective as a disinfection agent for ventilation systems, where residual viral matter can accumulate. In fact, the SARS virus was hypothesized to have been spread through the ventilation system in a Hong Kong hotel, one of the epicenters of the outbreak. Such an application would not only be highly useful to hotels and airlines, but could likely be implemented with a minimum of interference with current business practices.

Although the same limitations for ozone use in the hospitality and airline industries apply to hospitals and nursing homes, ozone use may be more feasible in hospital and nursing homes for a number of reasons. First of all, while hotels and airlines are susceptible to viral infection and spread, the odds of this occurring are statistically lower than the same risk in hospitals and nursing homes. Generally speaking, the people using airlines and hotels are relatively healthy, and it is the exception, rather than the rule, for a sick individual to utilize these industries. In hospitals, however, the reverse is true. Infected and immuno-compromised individuals congregate in hospitals, and accordingly increase the risk of viral infection and transmission.

Additionally, while hospitals and nursing homes are subject to greater risk of viral infection and spread, they are not always subject to the same economic pressures as the hospitality and airline industries for high turnover rates. It is true that hospitals and nursing homes often run full or even over-capacity, however it may still be possible to spend more time disinfecting rooms between patients. Finally, as with the hospitality and airline industries, hospitals may be able to find more limited applications for the use of ozone, such as the disinfection of ventilation systems.

There are other potential applications of ozone beyond these “core” industries. Because of the health hazards posed by ozone, these other applications include primarily non-occupied spaces or spaces that could be shut down for the evening or overnight and decontaminated while no one is present. A number of such applications are suggested above, including the decontamination of industrial fabric; the sterilization of medical equipment; the disinfection of areas such as granaries, sawmills, and research laboratories that use rodents; the decontamination of areas such as food processing factories and meat packing plants; the decontamination of barns, ranches and slaughterhouses; decontamination of large commercial buildings and various public facilities; and the decontamination of animal cages at zoos and veterinary offices. It is recommended that further research be conducted to determine both the efficacy of ozone for use in these areas, and also the level of corporate competition for ozone generators in these areas.

In conclusion, anecdotal studies have proven ozone to possess virucidal properties, however, these studies require further assessment as discussed. Despite ozone's potential risks, though, there is an increasingly strong need for a product with some of the characteristics of ozone: it's antiviral profile, its (relatively) short half-life, and its gaseous and diffusive nature. If ozone can be applied in a manner that greatly reduces its deleterious characteristics, while maintaining its strong antiviral profile, then ozone could well find a niche as an antiviral agent in a number of industries including hospitality, airline, health, packaging, and agriculture. Feasibility studies for each of these potential industries are also recommended. With the view that current global conditions encourage the emergence and spread of the new, hidden and/or resistant strains of viruses, ozone's application as an antiviral agent merits serious consideration.

### 6.0 DISCLAIMERS & LIMITATIONS

To facilitate the analysis of ozone as an antiviral agent in potential markets such as hospitality and airline industries, Treated Air Systems Manufacturing, Inc. retained BioStar Management, Inc. (BioStar) as consultants to assist in analyzing and preparing this report. The report presents the analysis on the anecdotal viral studies and potential market opportunities for ozone as antiviral agent.

This report is owned by Treated Air Systems Manufacturing, Inc. (TASM) and BioStar disclaims any undertaking or obligation to advise TASM, any company or person, of any change in any fact or matter impacting the opinions or views in the report, which may come or be brought to our attention in the future. BioStar has relied upon and assumed the completeness, accuracy and integrity of all information provided and obtained from public sources, for the report. Except as described in this report and subject to the exercise of its professional judgment, BioStar has not attempted to independently verify such completeness, accuracy and integrity. Accordingly, BioStar disclaims any responsibility to the completeness, accuracy and integrity of all information provided in the report obtained from public sources. BioStar disclaims any responsibility for the information from the report be:
1. quoted, summarized, paraphrased, excerpted or referred to, in whole or in part, in any circular, registration statement, prospectus or proxy statement, or in any report, document, filing, release or other written or oral communication prepared, issued or transmitted, and
2. relied upon by any other person or entity for any other purpose.

Specifically, Biostar excludes itself from liability in any way, shape, or form for misrepresentation, whether innocent, negligent, or fraudulent, or for any other action brought by TASM or a third party. TASM's receipt of this report and their remuneration of Biostar for the preparation and delivery of this report signifies acceptance of these terms. Should TASM fail to remunerate Biostar, they will be in breach of this contract, and accordingly Biostar will not be liable for any action arising from the use of this report, either by TASM or by a third party.

The statements made in this report are presented as suggestions and potential solutions, based on a broad survey of information available in the public domain. It is essential to be aware that further research is needed for every recommendation that Biostar has proposed, and that without said research, the true utility of these recommendations cannot be determined.

### 7.0 BIOSTAR MANAGEMENT INCORPARATED

BioStar Management, Inc is based in Vancouver, British Columbia. BioStar provides a broad range of consulting and management services to both emerging and established lifescience or biotechnology companies. Our services extend to lifescience investment firms, service firms, venture capital groups and brokerage firms. BioStar has many years of diverse experience in the biotechnology field through academia, research, business and consulting directly with a wide range of lifescience companies. Some of the company's specialty includes expertise in the fields of Biotechnology, Microbiology, Virology, Cell Biology, Genetics, Pathology, Diagnostics, Embryology, Medical Devices, and Immunology.

Our services for lifescience or biotechnology companies include: Independent advisory and expert opinions; Market analysis and market studies; Strategic Development Plan; Preparation of comprehensive budgets; Financial forecasts and projections; Preparation of business plan; Special project management and supervision; Our services for lifescience investment firms, service firms, venture capital groups and brokerage firms include: Scientific due diligence and assessment of the technology; Feasibility and market potential of the technology; Special reports; and Assessment of research plans, management team and budget analysis.

Our website address: www.biostarmanagement.ca

### 8.0 REFERENCES

Sunnen, 1994 Possible Mechanisms of Viral Inactivation by Ozone http://www.triroc.com/sunnen/topics/possiblemech.htm
Ishizaki K, Shinriki N, Matsuyama H. Inactivation of Bacillus spores by gaseous ozone. J Appl Bacteriol. 1986 January; 60(1):67-72. PMID: 3082844
Matus V, Nikava A, Prakopava Z, Konyew S: Effect of ozone on the survivability of Candida utilis cells. Vyestsi AkadNauuk Bssr Syer Biyal Navuk 1981; 0(3):49-52.
Matus V, Lyskova T, Sergienko I, Kustova A, Grigortsevich T, Konev V: Fungi; growth and sporulation after a single treatment of spores with ozone. Mikol Fitopatot 1982; 16(5):420-423.
Finch G R, Fairbairn N. Comparative inactivation of poliovirus type 3 and MS2 coliphage in demand-free phosphate buffer by using ozone. Appl Environ Microbiol. 1991 November; 57(11):3121-6. PMID: 1664198
Thraenhart O, Kuwert E. Comparative studies on the action of chlorine and ozone on polioviruses in the reprocessing of drinking water in Essen [Article in German]. Zentralbl Bakteriol [Orig B]. 1975 July; 160(4-5):305-41. PMID: 171885
Herbold K, Flehmig B, Botzenhart K. Comparison of ozone inactivation, in flowing water, of hepatitis A virus, poliovirus 1, and indicator organisms. Appl Environ Microbiol. 1989 November; 55(11):2949-53. PMID: 2560362
Vaughn J M, Chen Y S, Novotny J F, Strout D. Effects of ozone treatment on the infectivity of hepatitis A virus. Can J. Microbiol. 1990 August; 36(8):557-60. PMID: 2173968
Ivanova O E, Bogdanov M V, Kazantseva V A, Gabrilevskaia L N, Kodkind G Kh. Inactivation of enteroviruses in sewage with ozone. [Article in Russian] Vopr Virusol. 1983 November-December;28(6):693-8. PMID: 6322455
Vaughn J M, Chen Y S, Lindburg K, Morales D. Inactivation of human and simian rotaviruses by ozone. Appl Environ Microbiol. 1987 September; 53(9):2218-21. PMID: 2823709
Wells K H, Latino J, Gavalchin J, Poiesz B J. Inactivation of human immunodeficiency virus type 1 by ozone in vitro. Blood. 1991 Oct. 1; 78(7):1882-90. PMID: 1717074
Harakeh M, Butler M. Inactivation of human rotavirus, SA11 and other enteric viruses in effluent by disinfectants. J Hyg (Lond). 1984 August; 93(1):15763. PMID: 6086748
Akey D H, Walton T E. Liquid-phase study of ozone inactivation of Venezuelan equine encephalomyelitis virus. Appl Environ Microbiol. 1985 October; 50(4):882-6. PMID: 4083884
Katzenelson E, Koemer G, Biedermann N, Peleg M, Shuval H I. Measurement of the inactivation kinetics of poliovirus by ozone in a fast-flow mixer. Appl Environ Microbiol. 1979 April; 37(4):715-8. PMID: 36847
Carpendale M T, Freeberg J K. Ozone inactivates HIV at noncytotoxic concentrations. Antiviral Res. 1991 October; 16(3):281-92. PMID: 1805686
Emerson M A, Sproul O J, Buck C E. Ozone inactivation of cell-associated viruses. Appi Environ Microbiol. 1982 March; 43(3):603-8. PMID: 6280611
Jakab G J, Hmieleski R R. Reduction of influenza virus pathogenesis by exposure to 0.5 ppm ozone. J Toxicol Environ Health. 1988; 23(4):455-72. PMID: 3361616
Shin G A, Sobsey M D. Reduction of Norwalk Virus, Poliovirus 1, and Bacteriophage MS2 by Ozone Disinfection of Water. Appl Environ Microbiol. 2003 July; 69(7):3975-8. PMID: 12839770
Khadre M A, Yousef A E. Susceptibility of human rotavirus to ozone, high pressure, and pulsed electric field. J Food Prot. 2002 September; 65(9):1441-6. PMID: 12233855
Sato H, Wananabe Y, Miyata H. Virucidal effect of ozone treatment of laboratory animal viruses. Jikken Dobutsu. 1990 April; 39(2):223-9. PMID: 2163330
Wagner S J, Wagner K F, Friedman Li, Benade L F. Virucidal levels of ozone induce hemolysis and hemoglobin degradation. Transfusion. 1991 October; 31(8):748-51. PMID: 1926321
Murakami H, Mizuguchi M, Hattori M, Ito Y, Kawai T, Hasegawa J. Effect of denture cleaner using ozone against methicillin-resistant Staphylococcus aureus and E. coli Ti phage. Dent Mater J. 2002 March; 21 (1):53-60. PMID: 12046522s
Henderson F W, Dubovi E J, Harder S, Seal E Jr, Graham D. Experimental rhinovirus infection in human volunteers exposed to ozone. Am Rev Respir Dis. 1988 May; 137(5):11248. PMID: 2461669
Wolcott J A, Zee Y C, Osebold J W. Exposure to ozone reduces influenza disease severity and alters distribution of influenza viral antigens in murine lungs. Appl Environ Microbiol. 1982 September; 44(3):723-31. PMID: 6182839
Soukup J, Koren H S, Becker S. Ozone effect on respiratory syncytial virus infectivity and cytokine production by human alveolar macrophages. Environ Res. 1993 February; 60(2):178-86. PMID: 8472647
Jakab G J, Bassett D J. Influenza virus infection, ozone exposure, and fibrogenesis. Am Rev Respir Dis. 1990 May; 141(5 Pt 1):1307-15. PMID: 2339849
Jakab G J, Hmieleski R R. Reduction of influenza virus pathogenesis by exposure to 0.5 ppm ozone. J Toxicol Environ Health. 1988; 23(4):455-72. PMID: 3361616
Fairchild G A. Effects of ozone and sulfur dioxide on virus growth in mice. Arch Environ Health. 1977 January-February;32(1):28-33. PMID: 189703
De Medici D, Ciccozzi M, Fiore A, Di Pasquale S, Pariato A, Ricci-Bitti P, Croci L. Closed-circuit system for the depuration of mussels experimentally contaminated with hepatitis A virus. J Food Prot. 2001 June; 64(6):877-80. PMID: 11403143
Sunnen, 2003
SARS and Ozone Therapy: Theoretical Considerations http://www.triroc.com/sunnen/topics/sars.html
Lemmo, 2003
SARS: A place for aggressive naturopathic medicine http://www.lemmo.com/sars info.html
Kim J G, Yousef A E, Khadre M A. Ozone and its current and future application in the food industry. Adv Food Nutr Res. 2003; 45:167-218. PMID: 12402681
Agri-Food Surveillance Systems Branch: Newsletters, Vol. 1 No. 11 Jun. 2002 http://www.agric.qov.ab.ca/surveillance/snippets_v1n11june02.html
Kekez M M, Sattar S A. A new ozone-based method for virus inactivation: preliminary study. Phys Med. Biol. 1997 November; 42(11):2027-39. PMID: 9394395
TS03 Press Releases
www.tso3.com
Rice, 2002
Rice R G. Century 21—Pregnant with ozone. Ozone Science and Engineering 2002; 24:1-15

The invention claimed is:

1. A method of sterilizing a closed environment comprising:
(a) generating gaseous ozone into said closed environment to a predetermined ozone concentration;
(b) increasing the humidity of said closed environment;
(c) maintaining said predetermined ozone concentration for a predetermined period of time;
(d) after the expiry of said period of time, depleting said ozone;
(e) when said ozone concentration is reduced to a predetermined safe level, signalling.

2. An ozone generator comprising:
a humidifier;
a timer;
ozone generation means;
ozone depletion means;
movement means;
signalling means;
detection means for detecting ozone concentration and humidity of a close environment.

3. A method of inactivating a quantity of Norwalk virus in a closed environment, comprising:
(a) exposing the closed environment to an ozone concentration of 20 to 35 ppm for 30 to 70 minutes.

4. The method of claim 3 further comprising the step of:
(b) elevating the humidity of said closed environment while exposing the closed environment to said ozone concentration.
